SEQUENCE LISTING (2 sequences)

SEQ ID NO: 1 - DNA, 16 bp, Artificial Sequence
  Synthetic
aataatctcatgtcag

SEQ ID NO: 2 - DNA, 16 bp, Artificial Sequence
  Synthetic
ctttattcaatgtggc

(19) United States
(12) Patent Application Publication
(10) Pub. No.: US 2023/0357768 A1
(43) Pub. Date: Nov. 9, 2023

(54) PHARMACEUTICAL COMPOSITIONS COMPRISING AN ANTISENSE OLIGONUCLEOTIDE FOR ORAL ADMINISTRATION

(71) Applicants: ASTRAZENECA AB, Södertälje (SE); IONIS PHARMCEUTICALS, INC., Carlsbad, CA (US)

(72) Inventors: Anna TIVESTEN, Södertälje (SE); Nigel DAVIES, Södertälje (SE); Marie ELEBRING, Södertälje (SE); Peter GENNEMARK, Södertälje (SE); Mariagrazia MARUCCI, Södertälje (SE); Niclas CLEMMENSEN, Södertälje (SE); Hanna MATIC, Södertälje (SE); Okky PUTRA, Södertälje (SE); Pratik Pankaj UPADHYAY, Södertälje (SE); Katrin WALTER, Södertälje (SE); Lloyd TILLMAN, Carlsbad, CA (US); Luis DELLAMARY, Carlsbad, CA (US); Andreas RÅDEVIK, Södertälje (SE)

(21) Appl. No.: 18/245,609

(22) PCT Filed: Sep. 16, 2021
(86) PCT No.: PCT/EP2021/075548

§ 371 Date: Mar. 16, 2023

Related U.S. Application Data
(60) Provisional application No. 63/079,941, filed on Sep. 17, 2020.
(60) Provisional application No. 63/114,232, filed on Nov. 16, 2020.

(51) Int. Cl. C12N 15/113; A61P 3/06

(52) U.S. Cl. CPC C12N 15/113; A61P 3/06; C12N 2310/11

(57) ABSTRACT

A pharmaceutical composition comprising A) one or more ASO or a pharmaceutically acceptable salt thereof; B) one or more permeation enhancer; C) one or more optional pharmaceutically acceptable excipient; and D) one or more optional coating. Said composition for use in the treatment, prevention, or amelioration of a disease associated with PCSK9 or PNPLA3 in a subject.

### FIELD OF THE DISCLOSURE

The present disclosure relates to pharmaceutical compositions in a dosage form suitable for oral administration of antisense oligonucleotides or a pharmaceutically acceptable salt thereof and permeation enhancers, including methods of treatment using such formulations.

### BACKGROUND OF THE DISCLOSURE

Antisense oligonucleotides (hereinafter, ASOs) are synthetic oligonucleotides having a nucleobase sequence comprising between approximately 12 and 80 bases, that are complementary to a target mRNA. Unlike most conventional (small molecule and large molecule) therapies, ASOs can reach “undruggable” targets and enter the cytoplasm of a cell to downregulate target mRNA and thereby prevent the production of proteins involved in various disease processes. Thus, ASOs offer an exciting approach to the rational design of effective therapeutic products.

Only five ASOs are currently approved as therapeutic products for various disorders of which none are approved for oral delivery to target tissues outside the gastrointestinal tract. Oral delivery of ASOs is challenging as they are highly charged, hydrophilic macromolecules, having inherently poor intestinal stability and permeability and hence are expected to have negligible systemic bioavailability following oral administration (Maher, et al. Adv Drug Deli Rev, 106 (Pt B): 277-319 (2016)).

New chemistries developed, such as for example the constrained ethyl chemistry, have given ASOs improved gastrointestinal stability and potency. Furthermore, when the tissue target is in the liver, the tri-antennary N-acetyl galactosamine (GalNAc) chemistry promotes liver uptake via the asialoglycoprotein (ASGP) receptor primarily expressed on hepatocytes resulting in 10-30 fold increased potency in isolated hepatocytes, as well as in the liver in vivo (Biessen et al., Biochem J., 340 (PT 3): 783-792 (1999); Prakash et al., Nucleic Acids Res., 42: 8796-8807 (2014); Nair et al., J Am Chem Soc, 136:16958-16961 (2014); Crooke et al., Nucleic Acid Ther., (2018)).

When the target tissue for the ASO is the liver, delivery of potent, conjugated ASO's could benefit from delivery to the hepatic-portal vein so that first-pass hepatic extraction can be exploited. Direct delivery to the hepatic-portal vein is achieved following oral administration.

It has previously been shown that systemic bioavailability of ASOs following oral delivery can be achieved by co-formulating the ASO with a transient permeation enhancer (Tillman, et al., Journal of Pharmaceutical Sciences, 97(1): 225-236 (2008)). However, with the older ASO chemistries, therapeutic levels of ASOs in systemic circulation could only be achieved with pharmaceutical compositions containing very high doses of the ASO and the permeation enhancer, leading to non-viable pharmaceutical compositions that are inconvenient for patients and increase the risk of unwanted side effects. For example, systemic bioavailability of around 10% was demonstrated with 500 mg ASO with the 2′-O-(2-methoxyethyl) chemistry together and 3.5 g sodium caprate administered in 5 large capsules size 000 (Tillman, et al., Journal of Pharmaceutical Sciences, 97(1): 225-236 (2008)). Thus, there is a need to provide patients with an oral dosage form that delivers acceptable systemic bioavailability without compromising the therapeutic effect, safety and convenience.

### BRIEF SUMMARY OF THE DISCLOSURE

In order to achieve targeted delivery of ASOs to the liver, or hepatocytes at a therapeutically effective dose, the present disclosure provides ASOs comprising chemically modified oligonucleotides, such as constrained ethyl chemistry and liver targeting conjugates, such as GalNAc conjugation, in combination with permeation enhancers that facilitate systemic absorption following oral administration.

In accordance with the present disclosure, pharmaceutical compositions for oral administration and methods for treatment comprising administering the same are provided, wherein the pharmaceutical compositions for oral administration may be in a solid dosage form and comprise one or more ASOs, or a pharmaceutically acceptable salt thereof, and one or more permeation enhancers.

In certain embodiments, the present disclosure provides pharmaceutical compositions comprising conjugated ASOs. In certain embodiments, the present disclosure provides pharmaceutical composition comprising GalNAc conjugates of ASOs. In certain embodiments, the pharmaceutical compositions comprising conjugated ASOs results in increased delivery, uptake and activity in the liver and hepatocytes. In certain embodiments, the present disclosure provides pharmaceutical compositions comprising a conjugated ASO complementary to a nucleic acid transcript.

In certain embodiments, the present disclosure provides pharmaceutical compositions comprising ASOs comprising at least one modified sugar moiety. In certain embodiments, the present disclosure provides pharmaceutical compositions comprising ASOs comprising at least one sugar moiety having a 2′-OCH3 and/or at least one sugar moiety having a 2′-O(CH2)2OCH3. In certain embodiments, the present disclosure provides pharmaceutical compositions comprising ASOs comprising at least one sugar moiety having a constrained ethyl (cEt).

In certain embodiments, the present disclosure provides pharmaceutical compositions comprising a permeation enhancer selected from medium chain fatty acids and their salts. In certain embodiments, the present disclosure provides pharmaceutical compositions comprising a permeation enhancer that is sodium caprate. In certain embodiments, the present disclosure provides pharmaceutical compositions comprising Form A sodium caprate. As understood herein, “Form A sodium caprate” is understood to mean sodium caprate characterized by at least one of the following:
i) a wide-angle X-ray scattering (WAXS) spectrum which includes a peak at a region of 0.1 to 0.15 Å⁻¹;
ii) a wide-angle X-ray scattering (WAXS) spectrum which includes a peak at 0.12 and 0.23 Å⁻¹;
iii) a small-angle X-ray scattering (SAXS) spectrum which includes a peak at 0.12 and 0.23 Å⁻¹;
iv) an X-ray powder diffraction (XRPD) spectrum which includes a peak at 4° 2θ; or
v) a water content of less than about 3.5% as measured by Karl Fischer titration.

In certain embodiments, the present disclosure provides methods of treatment comprising administering the pharmaceutical compositions disclosed herein to a subject, wherein the pharmaceutical compositions comprise one or more ASOs, or a pharmaceutically acceptable salt thereof, and one or more permeation enhancers for oral administration of ASOs for the treatment of disease. In some embodiments, the oral delivery of ASOs reduce translation of the nucleic acid transcript to proteins involved in various disease processes. In some embodiments, the pharmaceutical compositions comprise one or more ASOs in a therapeutic effective amount to prevent, alleviate or ameliorate symptoms of a disease or to prolong the survival of the subject being treated. In other embodiments, the present disclosure provides pharmaceutical compositions comprising an ASO targeted to a PCSK9 nucleic acid, such as the ASOs described in International Patent Application No. PCT/US18/23936, filed Mar. 23, 2018, the disclosure of which is incorporated herein by reference. In other embodiments, the present disclosure provides pharmaceutical compositions comprising an ASO targeted to a PNPLA3 nucleic acid, such as the ASOs described in International Patent Application No. PCT/US19/051743, filed Sep. 18, 2019, the disclosure of which is incorporated herein by reference.

In some embodiments, methods are disclosed for treating, preventing, or ameliorating a disease associated with PCSK9 in a subject comprising administering to the subject a pharmaceutical composition comprising PCSK9 ASO. In certain embodiments, the subject has a cardiovascular disease. In certain embodiments, the disease is dyslipidemia. In certain embodiments, the disease is mixed dyslipidemia. In certain embodiments, the disease is hypercholesterolemia. Also disclosed are methods of reducing or inhibiting LDL-cholesterol levels and total cholesterol levels in a subject having, or at risk of having, a disease associated with PCSK9 comprising administering a pharmaceutical composition comprising PCSK9 ASO, thereby reducing or inhibiting LDL-cholesterol levels and total cholesterol levels in the subject.

In some embodiments, methods are disclosed for treating, preventing, or ameliorating a disease associated with PNPLA3 in a subject comprising administering to the subject a pharmaceutical composition comprising PNPLA3 ASO. In certain embodiments, the subject has a liver disease, non-alcoholic fatty liver disease (NAFLD), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. Also disclosed herein are methods of reducing or inhibiting liver damage, steatosis, liver fibrosis, liver inflammation, liver scarring or cirrhosis, liver failure, liver enlargement, elevated transaminases, or hepatic fat accumulation in a subject having, or at risk of having, a disease associated with PNPLA3 comprising administering a pharmaceutical composition comprising PNPLA3 ASO, thereby reducing or inhibiting liver damage, steatosis, liver fibrosis, liver inflammation, liver scarring or cirrhosis, liver failure, liver enlargement, elevated transaminases, or hepatic fat accumulation in the subject.

In certain embodiments, the pharmaceutical compositions herein comprise:
a) one or more ASO or a pharmaceutically acceptable salt thereof;
b) one or more permeation enhancer;
c) one or more optional pharmaceutically acceptable excipient; and
d) one or more optional coating.

In at least one embodiment, the pharmaceutical compositions comprise
a) one or more ASO or a pharmaceutically acceptable salt thereof present in an amount within the range from about 1 to about 100 mg;
b) one or more permeation enhancer present in an amount within the range from about 200 to about 1000 mg;
c) one or more pharmaceutically acceptable excipient present in an amount ranging from about 0 mg to about 600 mg; and
d) one or more optional coating present in an amount within the range from about 0 mg to about 200 mg.

In at least one embodiment, the pharmaceutical compositions comprise
a) one or more ASO that targets a PCSK9 nucleic acid or a pharmaceutically acceptable salt thereof present in an amount within the range from about 1 to about 100 mg;
b) sodium caprate present in an amount within the range from about 200 to about 1000 mg;
c) one or more pharmaceutically acceptable excipient present in an amount ranging from about 0 mg to about 600 mg; and
d) one or more optional coating present in an amount within the range from about 0 mg to about 200 mg.

In at least one embodiment, the pharmaceutical compositions comprise
a) ION-863633 or a pharmaceutically acceptable salt thereof present in an amount within the range from about 1 to about 100 mg;
b) Form A sodium caprate present in an amount within the range from about 200 to about 1000 mg;
c) one or more pharmaceutically acceptable excipient present in an amount ranging from about 0 mg to about 600 mg; and
d) one or more optional coating present in an amount within the range from about 0 mg to about 200 mg.

In at least one embodiment, the pharmaceutical compositions comprise
a) the sodium salt of ION-863633 present in an amount within the range from about 1 to about 100 mg;
b) Form A sodium caprate present in an amount within the range from about 200 to about 1000 mg;
c) one or more pharmaceutically acceptable excipient present in an amount ranging from about 0 mg to about 600 mg; and
d) one or more optional coating present in an amount within the range from about 0 mg to about 200 mg.

In at least one embodiment, the pharmaceutical compositions comprise
a) one or more ASO that targets a PCSK9 nucleic acid or a pharmaceutically acceptable salt thereof present in an amount within the range from about 1 to about 100 mg;
b) sodium caprate present in an amount within the range from about 200 to about 1000 mg;
c) one or more pharmaceutically acceptable excipient present in an amount ranging from about 0 mg to about 600 mg; and
d) one or more optional coating present in an amount within the range from about 0 mg to about 200 mg.

In at least one embodiment, the pharmaceutical compositions comprise
a) ION-975616 or a pharmaceutically acceptable salt thereof present in an amount within the range from about 1 to about 100 mg;
b) Form A sodium caprate present in an amount within the range from about 200 to about 1000 mg;
c) one or more pharmaceutically acceptable excipient present in an amount ranging from about 0 mg to about 600 mg; and
d) one or more optional coating present in an amount within the range from about 0 mg to about 200 mg.

In at least one embodiment, the pharmaceutical compositions comprise
a) the sodium salt of ION-975616 present in an amount within the range from about 1 to about 100 mg;
b) Form A sodium caprate present in an amount within the range from about 200 to about 1000 mg;
c) one or more pharmaceutically acceptable excipient present in an amount ranging from about 0 mg to about 600 mg; and
d) one or more optional coating present in an amount within the range from about 0 mg to about 200 mg.

### BRIEF DESCRIPTION OF THE DRAWINGS

FIG. 1 shows a flowchart for tablet production for a tablet comprising Form A sodium caprate and PCSK9 ASO (ION 863633).

FIG. 2 shows the dissolution profiles of tablets comprising Form A sodium caprate and PCSK9 ASO (ION 863633).

FIG. 3 shows the bioavailability of tablets comprising Form A sodium caprate and PCSK9 ASO (ION 863633) administered orally to dogs once daily after 4 weeks compared to subcutaneous injection.

FIG. 4 shows the dose of PCSK9 ASO (ION 863633) or rat specific tool ASO targeting Malat-1 administered to rats either as SC or IJ administration. (A) shows the liver concentration of unconjugated PCSK9 ASO (ION 863633) 48 h after dose versus dose levels of PCSK9 ASO (ION 863633); (B) shows liver concentration of unconjugated PCSK9 ASO (ION 863633) 48 h after dose versus dose levels of ION-704361; (C) shows the relative Malat-1 mRNA expression in the liver versus ION-704361 dose for SC and IJ administration; and (D) shows individual data from (C) plotted versus dose.

FIG. 5 shows the wide-angle X-ray scattering (WAXS) data for Form A sodium caprate.

FIG. 6 shows the small-angle X-ray scattering (SAXS) data for Form A sodium caprate.

FIG. 7 shows the XRPD pattern for Forms A sodium caprate

FIG. 8 shows the LDL-cholesterol reduction in healthy monkeys after repeated oral administration of PCSK9 ASO (ION 863633) tablets (Pre-dose-corrected LDL-cholesterol time profiles following repeated oral once daily dosing of AZD8823 with permeation enhancer for 14 days (N=2 per group). Data are relative to the average of two pre-dose values sampled two and one weeks before the start of treatment. Error bars denote standard error of the mean).

FIGS. 9A-B show the dissolution profiles of tablets comprising Form A sodium caprate and PNPLA3 ASO (ION 975616) of formulation 1 (FIG. 9A) and formulation 2 (FIG. 9B).

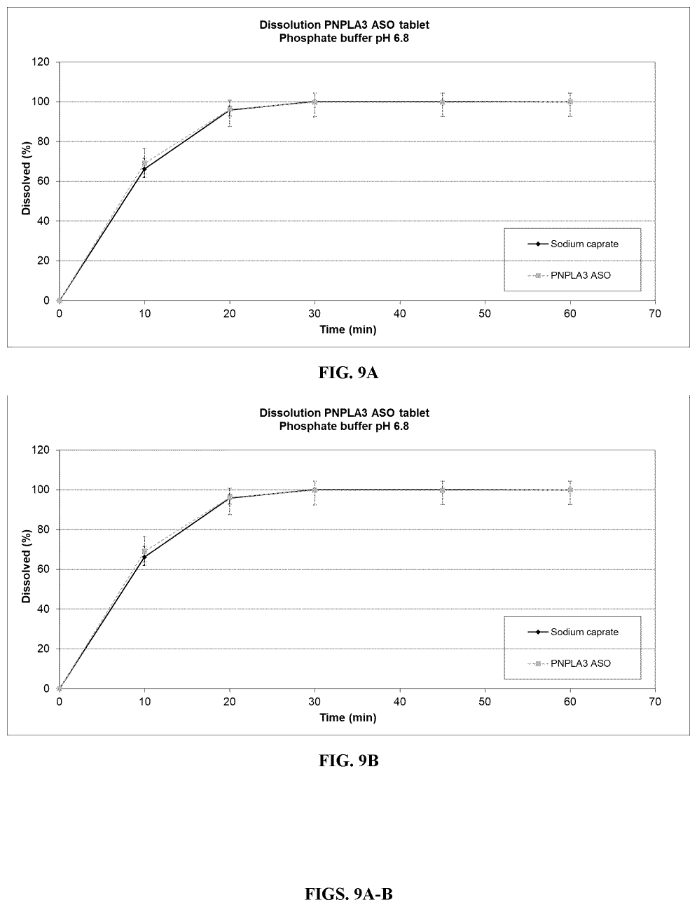

### DETAILED DESCRIPTION OF THE DISCLOSURE

The present disclosure provides pharmaceutical compositions and methods of use comprising administering one or more ASOs to a subject, such as conjugated ASOs, following oral administration. In certain embodiments, systemic bioavailability may be achieved via the oral administration of the presently disclosed pharmaceutical compositions comprising one or more ASOs. As used herein, “systemic bioavailability” is understood to mean the fraction of an orally administered dose of a compound that reaches the systemic circulation. In a further embodiment, enhanced target tissue bioavailability of ASOs, such as in the liver or the hepatocytes of the liver, relative to systemic exposure may be achieved via the oral administration of pharmaceutical compositions and ASOs provided herein. As used herein, “tissue bioavailability” is understood to mean the fraction of an orally administered dose of an ASO that reaches the target organ/cell. In certain embodiments, administration of the disclosed pharmaceutical compositions to a subject result in a liver bioavailability of up to 8%, such as up to 5%, and a productive bioavailability in hepatocytes of at least 30%. As used herein, “productive bioavailability” is understood to mean the fraction of an orally administered dose of an ASO that induces target engagement in the target organ/cell. As used herein, “target engagement” is understood to mean a pharmacodynamic effect, such as lowering of target protein.

The pharmaceutical compositions of the present disclosure may be in the form of a capsule or tablet, mini-tablet, pellet or granule; all of the above being collectively referred to as solid dosage forms and will comprise one or more ASO and one or more permeation enhancer. Mini-tablets, pellets or granules may be loaded into tablets or capsules or dispensed in sachets or other suitable means. In some embodiments, the solid dosage form is a capsule, it being understood herein that a capsule can include a liquid composition with a solid or semi-solid outer layer or can include completely solid compositions with a solid or semi-solid outer layer. In certain embodiments, the pharmaceutical compositions provided herein can be in the form of an immediate release, modified release, or delayed release solid dosage form.

In some embodiments, the pharmaceutical compositions comprise one or more ASOs in a therapeutically effective amount to prevent, alleviate or ameliorate symptoms of a disease or to prolong the survival of the subject being treated. As used herein, an “effective amount” is understood to mean the amount of an ASO(s), the administration of which to a subject, either in a single dose or as part of a series, is effective for treatment, i.e., to reduce the severity of a disease or disorder (or one or more symptoms thereof), ameliorate one or more symptoms of such a disease or disorder, prevent the advancement of such a disease or disorder, cause regression of such a disease or disorder, or enhance or improve the therapeutic effect(s) of another therapy. In certain embodiments, the amount of one or more ASOs in the pharmaceutical composition disclosed herein may range from about 1 mg to about 100 mg, for instance, from about 1 mg to about 40 mg, such as from about 5 mg to about 40 mg, and in at least one embodiment from about 50 mg to about 20 mg, such as 20 mg. The amount of ASO(s) in the pharmaceutical formulations disclosed relates to the amounts to be administered in a single dosage unit but may be divided to form mini-tablets, pellets or granules. For instance, in certain embodiments, the present disclosure relates to mini-tablets, pellets or granules wherein each pellet or granule within the tablet or capsule has a percentage of ASO that, when the weights of each ASO in each pellet or granule is added together, equal an amount ranging from about 1 mg to about 100 mg, for instance, from about 1 mg to about 20 mg, such as from about 1 mg to about 10 mg, and in at least one embodiment from about 1 mg to about 5 mg, such as 3 mg.

In some embodiments, the pharmaceutical compositions disclosed herein comprise one or more permeation enhancers in an amount to achieve systemic exposure. In certain embodiments, the amount of one or more permeation enhancers may range from about 200 mg to about 1500 mg, for instance from about 500 mg to about 1000 mg, for example about 650 mg to about 850 mg, such as from about 700 mg to about 800 mg, and in at least one embodiment about 700 mg. The amount of permeation enhancer in the pharmaceutical formulations disclosed relates to the amounts to be administered in a single dosage unit but may be divided to form mini-tablets, pellets or granules. For instance, in certain embodiments, the present disclosure relates to mini-tablets, wherein each pellet or granule within the tablet or capsule has a percentage of permeation enhancers that, when the weights of each permeation enhancer in each pellet or granule is added together, equal an amount ranging from about 200 mg to about 1500 mg, for instance from about 500 mg to about 1000 mg, for example about 650 mg to about 850 mg, such as from about 700 mg to about 800 mg, and in at least one embodiment about 700 mg.

The pharmaceutical compositions of the disclosure will optionally further include one or more acceptable pharmaceutical excipients to enable manufacture and influence the performance/function of the solid dosage form such as a capsule, tablet, mini-tablet, pellet, or granule. Accordingly, in one or more embodiments, the pharmaceutical compositions comprise one or more ASO, one or more permeation enhancer, and one or more pharmaceutically acceptable excipient. In some embodiments, the one or more pharmaceutically acceptable excipient is chosen from diluents/fillers, anti-tacking agents, emulsifiers, lubricants, flow agents/glidants, disintegrants, plasticizers, solubilizers, solvents and binders. In some embodiments, the solid dosage of the pharmaceutical compositions may further comprise one or more optional coatings, such as functional coating, for instance, an outer protective gastro-resistant coating.

The pharmaceutical compositions of the present disclosure can be prepared by a variety of processes and order of addition of excipients. Solid dosage forms may be manufactured by wet granulation, dry granulation, direct blending, tableting, capsule filling, coating procedures or any other pharmaceutically acceptable process as well as mixing and drying steps if/as needed. The utility of these pharmaceutical compositions is not limited to a specific dosage form or manufacturing process.

### Antisense Oligonucleotides

ASOs that can be orally administered are provided herein. Exemplary ASOs used in the pharmaceutical compositions of the present disclosure may comprise one or more modifications, for example, the ASOs may comprise one or more modified internucleoside linkage, a modified sugar, and/or a modified nucleobase. In other embodiments, the ASOs may incorporate a conjugate group. In certain embodiments, the ASOs comprise multiple modifications. It is understood that the sequence set forth in any SEQ ID NO in the examples contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Compounds described by ION number indicate a combination of nucleobase sequence, chemical modification, motif, and/or conjugate.

In certain embodiments, the modified oligonucleotide comprises at least one modified internucleoside linkage, such as a phosphorothioate internucleoside linkage.

In one or more embodiment, the ASO comprises at least one modified sugar. In certain embodiments, the at least one modified sugar comprises a 2′-OCH3 (“OMe” or “O-methyl”), and/or a 2′-O(CH₂)₂OCH₃ (“MOE”). In other embodiments, the at least one modified sugar is cEt modified sugar moiety, where “cEt” or “constrained ethyl” means a bicyclic furanosyl sugar moiety comprising a bridge connecting the 4′-carbon and the 2′-carbon, wherein the bridge has the formula: 4′-CH(CH₃)—O-2′. In certain embodiments, the ASO comprise a mixture of modified sugars, for example, an ASO comprising at least one 2′-O-methoxyethyl group (MOE) and at least one cEt modified sugar moiety.

In certain embodiments, the ASO comprises at least one modified nucleobase, such as 5-methylcytosine.

The ASOs used in the pharmaceutical compositions of the present disclosure may further incorporate a conjugate group. In certain embodiments, conjugate groups modify one or more properties of the attached ASO, including but not limited to pharmacodynamics, pharmacokinetics, stability, binding, absorption, tissue distribution, cellular distribution, cellular uptake and clearance.

In certain embodiments, the ASO can be conjugated to a ligand to target a receptor expressed on the surface of a cell. In certain embodiments, the ligand promotes distribution of the ASO to the liver. In other embodiments, the ligand promotes uptake within the hepatocytes or other cells of the of the liver. In certain embodiments, the conjugate is a polysaccharide, a vitamin, an antibody, a peptide or aptamer, or other ligands for receptors expressed on liver cells including but not limited to transferrin and low-density lipoprotein receptors. In one or more embodiment, the ligand is for the asialoglycoprotein receptor expressed on hepatocytes. In certain embodiments, the ligand is N-acetylgalactoseamine (GalNAc) capable of interacting with the asialoglycoprotein receptor expressed on hepatocytes.

Conjugate groups may consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

In one or more embodiment, the conjugate group is linked to the ASO at the 5′ end of the ASO. In other embodiments, the conjugate group is linked to the ASO at the 3′ end of the ASO. In certain embodiments, the conjugate group comprises one or more GalNAc sugar units, at least two GalNAc sugar units, or at least three GalNAc sugar units.

The ASOs of the present disclosure can be 12 to 80, 14 to 80, 16 to 80, 16 to 50, 16 to 30, 17 to 80, 17 to 50, 17 to 30, 18 to 80, 18 to 50, 18 to 30, 19 to 80, 19 to 50, 19 to 30, 20 to 80, 20 to 50, or 20 to 30 linked nucleosides in length. In one or more embodiments, the ASOs can be 12-30 linked nucleosides, for instance, the modified ASO can be 16-25 linked nucleosides, and in one or more embodiment, 16 linked nucleosides.

In one or more embodiments, the ASO comprises a nucleobase sequence of AATAATCTCATGTCAG (SEQ ID NO: 1). In one or more embodiments, the ASO comprises a nucleobase sequence of CTTTATTCAATGTGGC (SEQ ID NO: 2).

In certain embodiments, the ASO comprises or consists of a modified oligonucleotide 12-80 linked nucleobases in length having a nucleobase sequence comprising the sequence of SEQ ID NO: 1, wherein the modified oligonucleotide comprises
a gap segment consisting of at least ten linked deoxynucleosides;
a 5′ wing segment consisting of three linked nucleosides; and
a 3′ wing segment consisting of three linked nucleosides;
   wherein the gap segment is positioned between the 5′ wing segment and the 3′ wing segment, wherein each nucleoside of each wing segment comprises a cEt sugar; wherein each internucleoside linkage is a phosphorothioate linkage and wherein each cytosine is a 5-methylcytosine.

In certain embodiments, the ASO comprises or consists of a modified oligonucleotide 12-80 linked nucleobases in length having a nucleobase sequence comprising the sequence of SEQ ID NO: 2, wherein the modified oligonucleotide comprises
a gap segment consisting of at least ten linked deoxynucleosides;
a 5′ wing segment consisting of three linked nucleosides; and
a 3′ wing segment consisting of three linked nucleosides;
   wherein the gap segment is positioned between the 5′ wing segment and the 3′ wing segment, wherein each nucleoside of each wing segment comprises a cEt sugar; wherein each internucleoside linkage is a phosphorothioate linkage and wherein each cytosine is a 5-methylcytosine.

In certain embodiments, the pharmaceutical compositions provided herein comprise a pharmaceutically acceptable salt of the ASO. In certain embodiments, the salt is a sodium salt. In certain embodiments, the salt is a potassium salt.

In certain embodiments, the one or more ASO targets a PCSK9 nucleic acid. As used herein, the term “PCSK9 nucleic acid” means any nucleic acid encoding PCSK9. For example, in certain embodiments, a PCSK9 nucleic acid includes a DNA sequence encoding PCSK9, an RNA sequence transcribed from DNA encoding PCSK9 (including genomic DNA comprising introns and exons) and an mRNA sequence encoding PCSK9. “PCSK9 mRNA” means an mRNA encoding a PCSK9 protein. The target may be referred to in either upper or lower case. In certain embodiments, the one or more ASO targets a PNPLA3 nucleic acid. As used herein, the term “PNPLA3 nucleic acid” means any nucleic acid encoding PNPLA3. For example, in certain embodiments, a PNPLA3 nucleic acid includes a DNA sequence encoding PNPLA3, an RNA sequence transcribed from DNA encoding PNPLA3 (including genomic DNA comprising introns and exons) and an mRNA sequence encoding PNPLA3. “PNPLA3 mRNA” means an mRNA encoding a PNPLA3 protein. The target may be referred to in either upper or lower case.

In certain embodiments of the present disclosure, one or more ASO includes the ASOs described in International Patent Application No. PCT/US18/23936. For example, the ASO(s) is chosen from ION 863633 and ION 848833, or a salt of either ION 863633 and ION 848833, and combinations of ION 863633 and ION 848833 and salts thereof. In at least one embodiment, the ASO comprises ION 848833. In another embodiment, the ASO comprises a salt of ION 848833. In yet another embodiment, the ASO comprises a sodium salt of ION 848833. In certain embodiments of the present disclosure, one or more ASO includes the ASOs described in International Patent Application No. PCT/US19/051743. For example, the ASO(s) is chosen from ION 975616 and ION 916333, or a salt of either ION 975616 and ION 916333, and combinations of ION 975616 and ION 916333 and salts thereof. In at least one embodiment, the ASO comprises ION 975616. In yet another embodiment, the ASO comprises a salt of ION 975616. In another embodiment, the ASO comprises a sodium salt of ION 975616.

In certain embodiments, the one or more ASO described herein is modified and further comprises a conjugate group. In certain embodiments, the modified ASOs comprise a gapmer or fully modified motif and a conjugate group comprising one or more, two, or three GalNAc ligands. In yet another embodiment, the ASO described herein comprises or consists of an ASO targeted to a PCSK9 nucleic acid that is further conjugated to one or more GalNAc and comprises a gap segment consisting of ten linked deoxynucleosides;
a 5′ wing segment consisting of three linked nucleosides; and
a 3′ wing segment consisting of three linked nucleosides;
wherein the gap segment is positioned between the 5′ wing segment and the 3′ wing segment, wherein each nucleoside of each wing segment comprises a cEt sugar; wherein each internucleoside linkage is a phosphorothioate linkage and wherein each cytosine is a 5-methylcytosine. In yet another embodiment, the ASO described herein comprises or consists of an ASO targeted to a PNPLA3 nucleic acid that is further conjugated to one or more GalNAc and comprises a gap segment consisting of ten linked deoxynucleosides;
a 5′ wing segment consisting of three linked nucleosides; and
a 3′ wing segment consisting of three linked nucleosides;
   wherein the gap segment is positioned between the 5′ wing segment and the 3′ wing segment, wherein each nucleoside of each wing segment comprises a cEt sugar; wherein each internucleoside linkage is a phosphorothioate linkage and wherein each cytosine is a 5-methylcytosine.

In one or more embodiment, the one or more ASO comprises or consists of ION 863633 or salt thereof, having the following chemical structure (SEQ ID NO: 1):

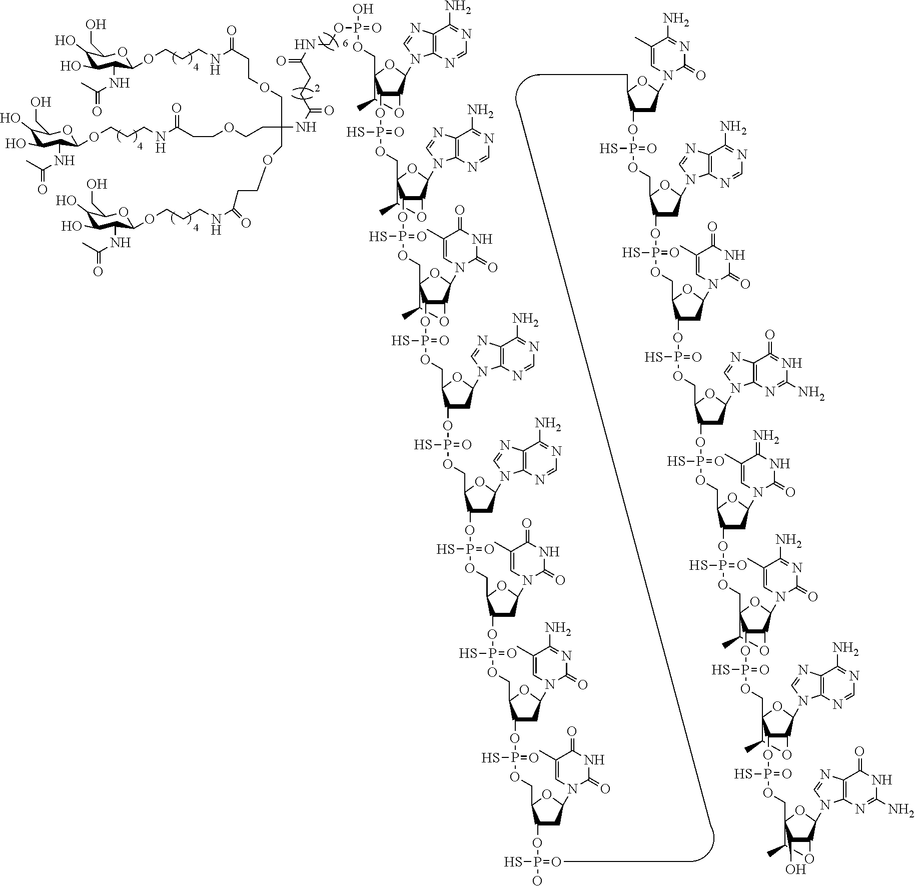

In other embodiment, the ASO comprises or consists of ION 863633, or salt thereof, having the following chemical structure (SEQ ID NO: 1):

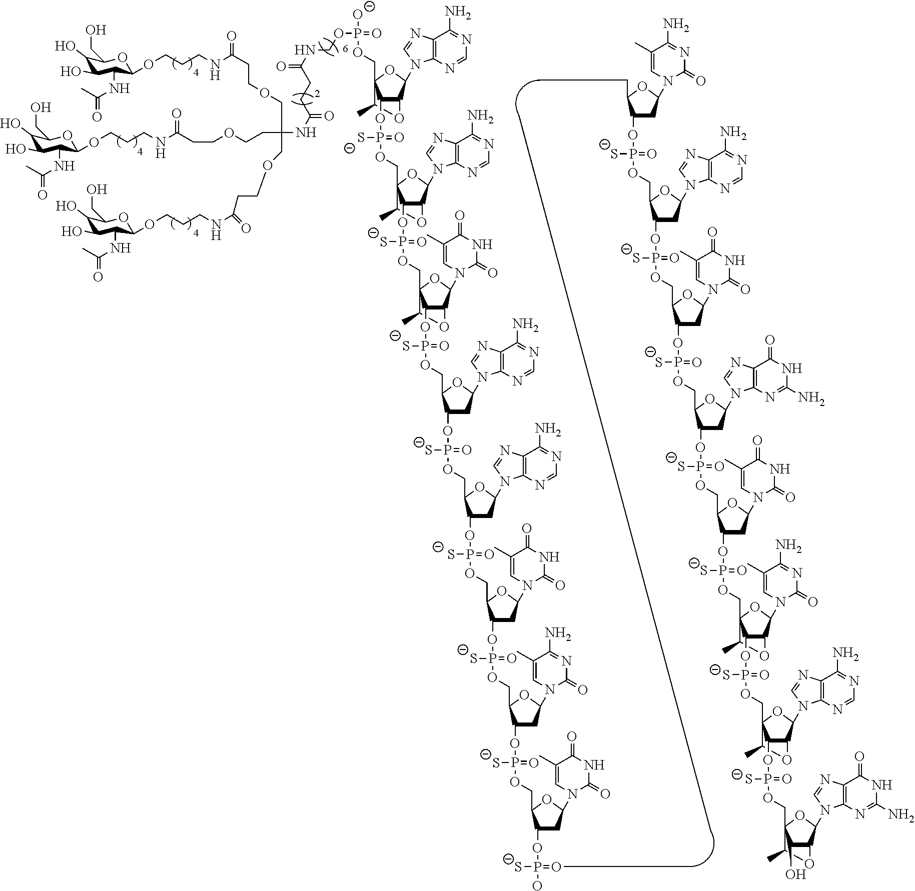

In certain embodiments, the ASO comprises or consists of the sodium salt of ION 863633, having the following chemical structure (SEQ ID NO: 1):

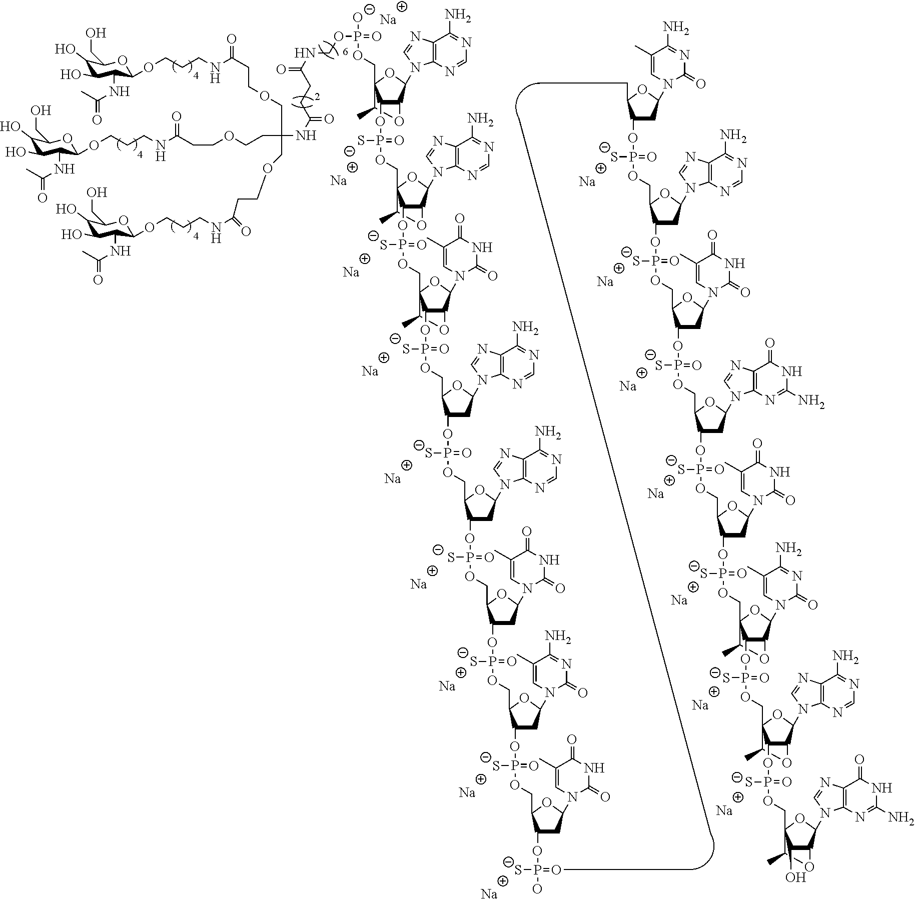

In one or more embodiment, the one or more ASO comprises or consists of ION 975616 or salt thereof, having the following chemical structure (SEQ ID NO: 2):

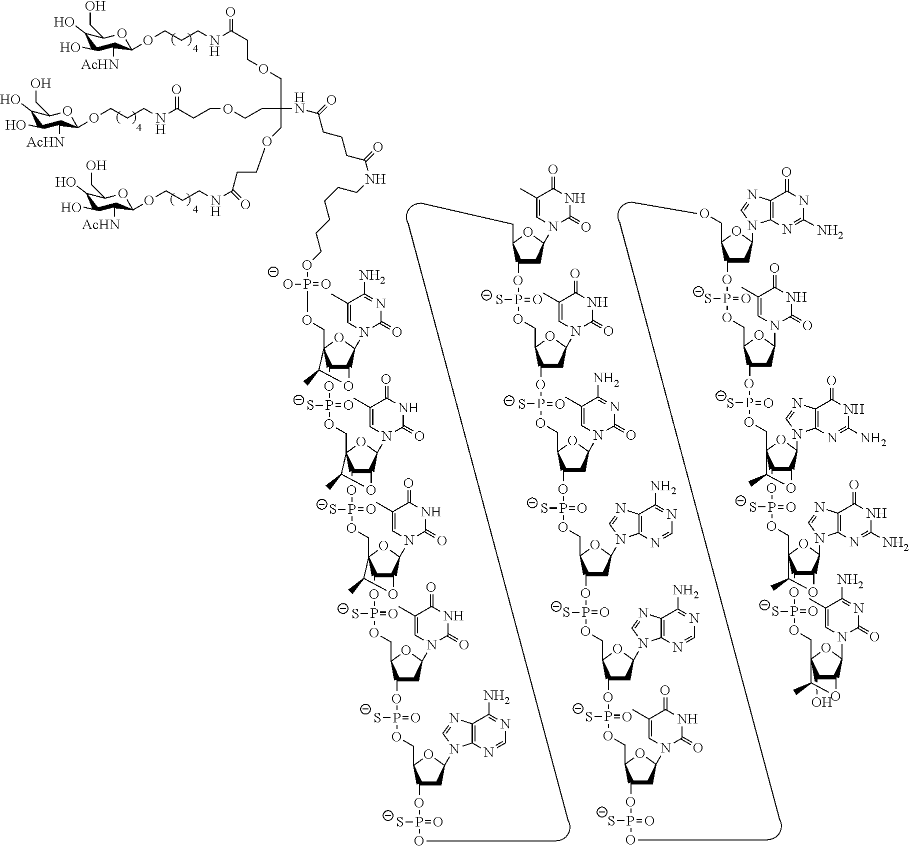

In certain embodiments, the one or more ASO comprises or consists of the sodium salt of ION 975616, having the following chemical structure (SEQ ID NO: 2):

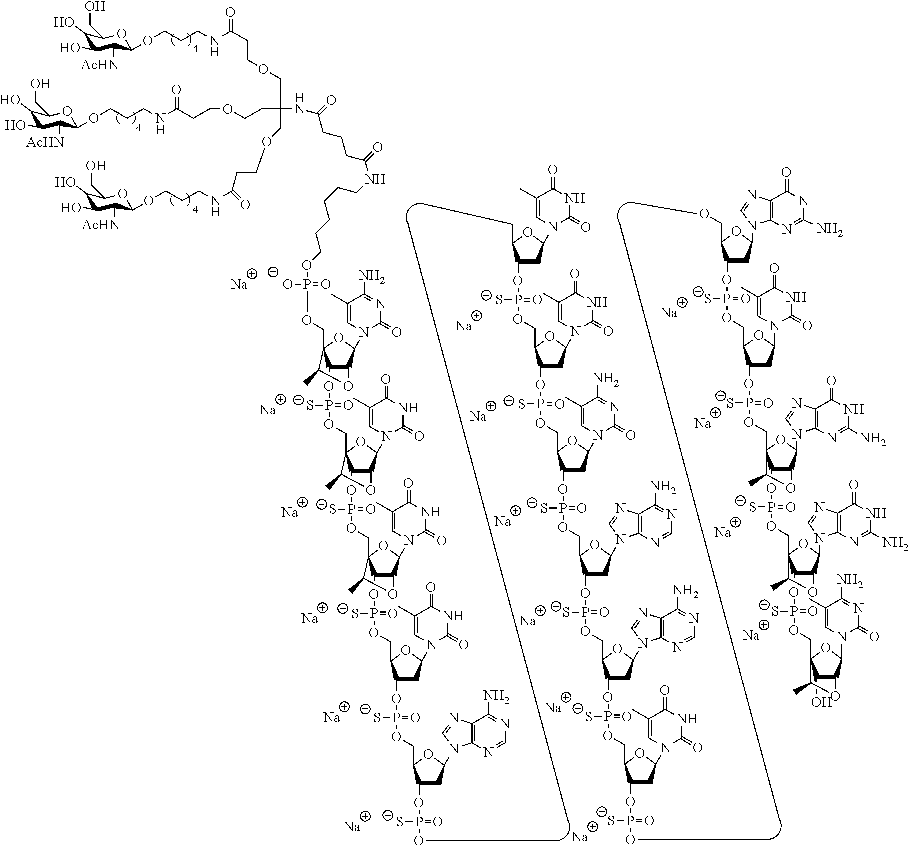

In one or more embodiment, the one or more ASO comprises or consists of ION 975616 or salt thereof, having the following chemical structure (SEQ ID NO: 2):

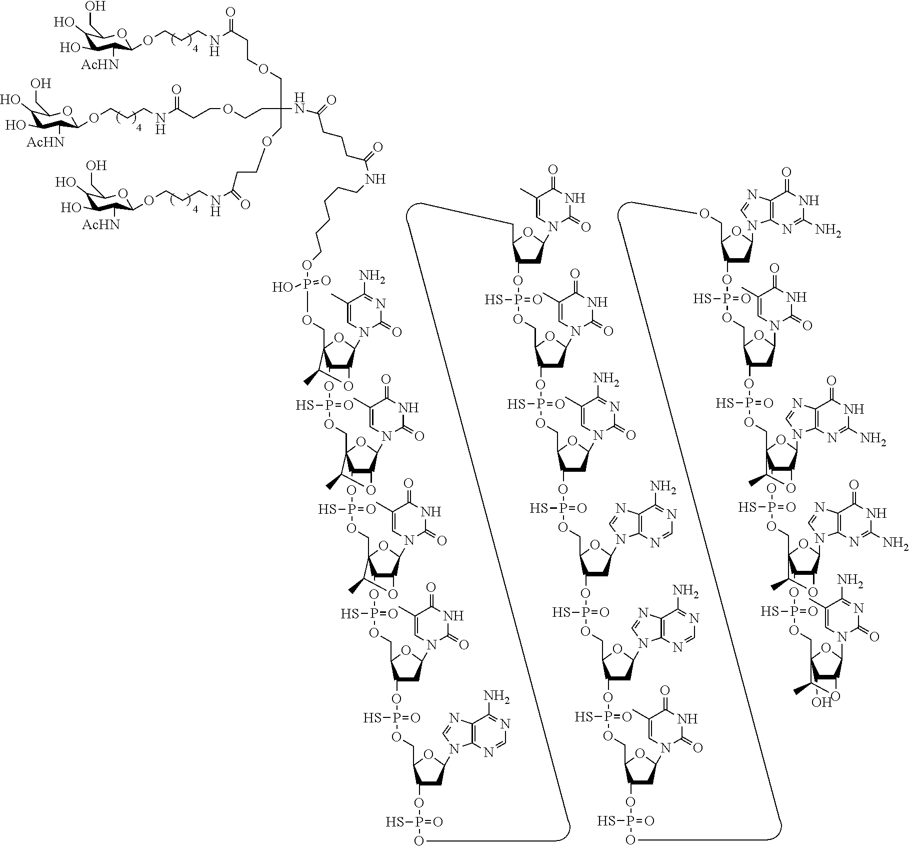

In any of the foregoing embodiments, the ASO can be at least 85%, at least 90%, at least 95%, at least 98%, at least 99%, or 100% complementary to a nucleic acid encoding PCSK9.

The pharmaceutical compositions described herein can be formulated for a particular solid dosage form. Dosage regimens can be adjusted to provide the optimum response. It can be useful to formulate compositions in dosage unit forms for ease of administration and uniformity of dosage. Dosage unit forms as used herein refers to physically discrete units suited as unitary dosages for the subjects to be treated; each unit contains a predetermined quantity of one or more ASO or pharmaceutically acceptable salt thereof calculated to produce a therapeutic effect in association with the required pharmaceutical carrier. For example, the pharmaceutical compositions disclosed herein can comprise a dose of one or more ASO or a pharmaceutically acceptable salt thereof in an amount ranging from about 0.1 mg to about 100 mg and in some embodiments, from about 0.5 mg to about 40 mg, for instance, from about 1 mg to about 40 mg, such as about 5 mg to about 40 mg. In some embodiments, the one or more ASO is present in an amount ranging from about 0.1 mg, about 0.2 mg, about 0.3 mg, about 0.4 mg, about 0.5 mg, about 0.6 mg, about 0.7 mg, about 0.8 mg, about 0.9 mg, about 1mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, and about 100 mg. In some embodiments, the one or more ASO or a pharmaceutically acceptable salt thereof is in an amount ranging from about 0.1% to about 12% by weight of the solid dosage form.

### Permeation Enhancers

Examples of permeation enhancers suitable for use herein include, but are not limited to, medium chain fatty acids (C_{6.2O}) and their salts, esters or ethers; derivatives of medium chain fatty acids; medium chain mono-, di- and tri glycerides and derivatives thereof; polyoxylglycerides; acylated amino acids; organic acids; acyl carnitines; alkyl saccharides; bile salts; aromatic alcohols, chelating agents, polymers, mixed micelles, reversed micelles, and self-emulsifying systems (e.g., SEDDS, SMEDDS, or SNEDDS); together with mixtures and combinations thereof.

Non-limiting examples of the various types of permeation enhancers are listed in the Table 1 below. Many of these permeation enhancers may be available in several different brands and qualities and mixtures thereof.

TABLE 1
Permeation Enhancers

| Permeation enhancer type | Examples |
|---|---|
| Medium chain fatty acids and their | Sodium caprylate, sodium caprate, sodium laurate, |
| salts | sodium myristate, sodium palmitate and sodium |
| | stearate. |
| Derivatives of fatty acids or | N-[8-(2-hydroxybenzoyl) amino] caprylate (SNAC), |
| acylated amino acids | 8-(N-2-hydroxy-5-chloro-benzoyl)-amino-caprylate |
| | (5-CNAC) and N-(4-chlorosalicyloyl)-4- |
| | aminobutyrate (4-CNAB) |
| Medium chain mono-, di-and tri | Caprylic mono-, di-and tri-glyceride, capric mono-, |
| glycerides and mixtures thereof | di-and tri-glyceride, glyceryl caprylate and glyceryl |
| | caprate |
| Polyoxylglycerides | Propyleneglycol monocaprylate, propyleneglycol |
| | monocaprate, caprylocaproyl polyoxyglycerides, |
| | lauroyl polyoxyglycerides (e.g., LABRASOL ®)), |
| | polyoxyethylene glycerol fatty acid esters, |
| | polyoxyethylene mono-and di-glycerides, macrogol |
| | glycerides and polyoxyethlene lauryl ether |
| Organic acids and salts thereof | Citric acid, tartaric acid, gluconic acid, oxalic acid, |
| | geranic acid and malic acid |
| Acyl carnitines | Lauroyl-L-carnitine, myristoyl carnitine and |
| | palmitoyl carnitine |
| Alkyl saccharides | N-octyl-beta-D-glucopyranoside, n-dodecyl-beta-D- |
| | maltoside, tridecyl-beta-D-maltoside, decanoyl-N- |
| | methyl glucamine and sucrose esters such as sucrose |
| | laurate |
| Bile acids and salts thereof | Chenodeoxycholic acid, ursodeoxycholic acid, |
| | taurochenodeoxycholic acid, glycodeoxycholic acid |
| | taurocholic acid, glycocholic acid and cholic acid, |
| Aromatic alcohols | Propyl gallate |
| Chelating agents | Ethylenediaminetetraacetic acid |
| Polymers | Polycarbophils/carbomers, chitosan and derivatives, |
| | thiolated polymers |

In certain embodiments, the present disclosure provides pharmaceutical compositions comprising Form A sodium caprate. Form A sodium caprate may be characterized by at least one of the following:
i) a wide-angle X-ray scattering (WAXS) spectrum which includes a peak at a region of 0.1 to 0.15 Å⁻¹;
ii) a wide-angle X-ray scattering (WAXS) spectrum which includes a peak at 0.12 and 0.23 Å⁻¹;
iii) a small-angle X-ray scattering (SAXS) spectrum which includes a peak at 0.12 and 0.23 Å⁻¹;
iv) an X-ray powder diffraction (XRPD) spectrum which includes a peak at 4° 2θ; or
v) a water content of less than about 3.5% as measured by Karl Fischer titration.

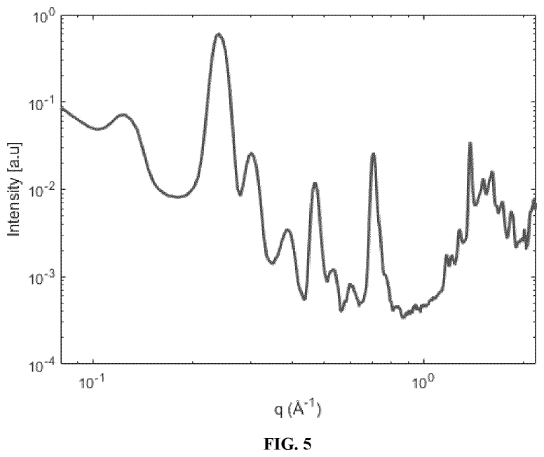

In some embodiments, Form A sodium caprate is identified by small-angle X-ray scattering (SAXS). In some embodiments, a sodium caprate form is identified by wide-angle X-ray scattering (WAXS). SAXS and WAXS are scattering techniques in which X-rays are scattered by fluctuations in the electron density in the sample. Thus, in some embodiments, SAXS and WAXS are used to determine the crystalline structure. SAXS typically diffracts at a smaller angle than WAXS (i.e., the distance between the sample and detector is longer for SAXS than WAXS). Methods of preparing a SAXS or WAXS experimental set-up are known to the skilled artisan. FIG. 5 shows the wide-angle X-ray scattering (WAXS) spectrum for Form A sodium caprate.

In some embodiments, Form A sodium caprate comprises a WAXS peak at a region of about 0.1 to about 0.15 Å⁻¹. In some embodiments, Form A sodium caprate comprises more than one WAXS peaks at a region of about 0.12 to about 0.23 Å⁻¹. In some embodiments, Form A sodium caprate comprises a WAXS peak at about 0.12 Å⁻¹. In some embodiments, Form A sodium caprate comprises a WAXS peak at about 0.23 Å⁻¹;.

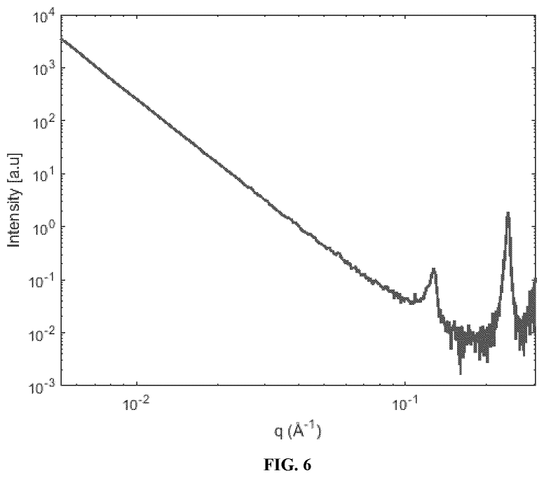

In some embodiments, Form A sodium caprate comprises a SAXS peak at a region of about 0.1 to about 0.15 Å⁻¹. In some embodiments, Form A sodium caprate comprises more than one SAXS peaks at a region of about 0.12 to about 0.23 Å⁻¹. In some embodiments, Form A sodium caprate comprises a SAXS peak at about 0.12 Å⁻¹. In some embodiments, Form A sodium caprate comprises a SAXS peak at about 0.23 Å⁻¹. FIG. 6 shows the small-angle X-ray scattering (SAXS) spectra for Form A sodium caprate.

In some embodiments, Form A sodium caprate form is identified by X-ray powder diffraction (XRPD). XRPD is a diffraction method, i.e., scattering from atoms in planes in an ordered crystal lattice. In general, XRPD can be used to detect unique fingerprints of crystallographic unit cells present within a crystalline substance, with each type of unit cell appearing as a peak in a particular position on an XRPD pattern. Thus, crystalline substances may be distinguished by their unit cells via identification of the peaks appearing on the diffraction pattern. Methods of preparing a XRPD experimental set-up are known to the skilled artisan.

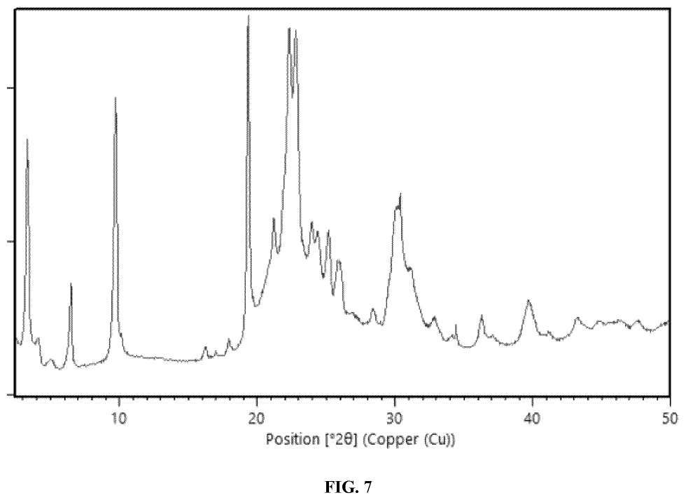

In some embodiments, Form A sodium caprate has an XRPD pattern substantially as shown in the FIG. 7. The term “substantially as shown in” when referring, for example, to an XRPD pattern, refers to a pattern that is not necessarily identical to those depicted herein, but that falls within the limits of experimental error or deviations when considered by one of ordinary skill in the art. Various values for XRPD are described herein. As used throughout the present disclosure (unless explicitly noted), all XRPD peak position values are to be construed to be ±0.5° 2θ. In some embodiments, Form A sodium caprate comprises a XRPD peak at about 4° 2θ.

In some embodiments, the water content of Form A sodium caprate is determined by Karl Fischer titration. Karl Fischer titration uses coulometric or volumetric titration to determine trace amounts of water in a sample. Methods of performing Karl Fischer titration are known to the skilled artisan. In some embodiments, Form A sodium caprate may be characterized by a water content by Karl Fischer titration below 2%, below 1.9%, below 1.8%, below 1.7%, below 1.6%, below 1.5%, or below 1.4%. In some embodiments, Form A sodium caprate has a water content of about 0.4% to about 2.0%. In some embodiments, Form A sodium caprate has a water content of about 1%, about 1.1%, about 1.2%, about 1.3%, about 1.4%, about 1.5%, about 1.6%, about 1.7%, about 1.8%, about 1.9%, or about 2.0%.

The pharmaceutical compositions disclosed herein can comprise one or more permeation enhancer in an amount ranging from about 200 mg to about 1500 mg. In some embodiments, the permeation enhancer(s) is present in an amount ranging from about 500 mg to about 1000 mg, such as about 525 mg, about 550 mg, about 575 mg, about 600 mg, about 625 mg, about 650 mg, about 675 mg, about 700, mg, about 725 mg, about 750 mg, about 775 mg, about 800 mg, about 825 mg, about 850 mg, about 875 mg, about 900 mg, about 925 mg, about 950 mg, about 975 mg, and about 1000 mg.

### Pharmaceutical Acceptable Excipients

The pharmaceutical compositions of the present disclosure may further comprise one or more pharmaceutically acceptable excipient. In certain embodiments, the pharmaceutically acceptable excipients may be any compound or mixture of compounds that is added to the pharmaceutical compositions that is suitable for oral delivery. Pharmaceutically acceptable excipients are well known in the art and any selection depends on the intended use and method of administration of the pharmaceutical compositions. A person skilled in the art may select one or more of the pharmaceutically accepted excipients with respect to the particular desired properties of the solid oral dosage form. Pharmaceutically acceptable excipient include for example diluents/fillers, anti-tacking agents, emulsifiers, lubricants, flow agents/glidants, disintegrates, compression aids, binders, plasticizers, solubilizers, solvents, and permeation enhancers other than the permeation enhancers already required in the disclosed pharmaceutical compositions herein. Pharmaceutical acceptable excipients suitable for use herein include, but are not limited to, examples listed below. Each excipient may be available in several different brands and qualities and mixtures thereof.

Non-limiting examples of pharmaceutically acceptable excipients include microcrystalline cellulose, dicalcium phosphate, lactose, mannitol, sodium stearyl fumarate (PRUV), magnesium stearate, silica colloidal hydrated, crospovidone, sodium croscarmellose, sodium bicarbonate, low-substituted hydroxypropylcellulose (L-HPC), sodium starch glycolate, water, ethanol, isopropyl alcohol or other solvents, polyvinylpyrrolidone (PVP), hydroxy propyl cellulose (HPC), hydroxypropylmethylcellulose (HPMC), (tromethamine) (TRIS), any salt of carbonate, borate, phosphate, tartaric acid, magnesium hydroxide, magnesium oxide, sodium bicarbonate, propyl gallate, alpha-tocopherol, butylated hydroxy anisole (BHA), ascorbic acid, solutol, polysorbate 80, and ethylenediaminetetraacetic acid (EDTA).

The amount of the excipients in the presently disclosed pharmaceutical compositions may vary within ranges conventional in the art. The pharmaceutically acceptable excipients may be present in the pharmaceutical compositions disclosed herein in an amount ranging from about 0.1 mg to about 600 mg. In certain embodiments, the amount of excipient may be expressed as percent by weight of solid dosage form. For instance, in some embodiments, the pharmaceutical compositions disclosed herein may comprise excipients ranging from about 0.001% to about 50% by weight of the solid dosage form.

### Coatings

As provided herein, the pharmaceutical compositions may be an immediate, modified or delayed release formulation. Exemplary modified or delayed release formulations of the present disclosure may include one or more gastro-resistant coating, for example, an outer gastro-resistant or semi-permeable coating which may include an aqueous/organic solvent based coating polymer, such as Hypromellose acetate succinate (HPMCAS), or methacrylic acid copolymers (e.g., EUDRAGIT®), specifically those sold under the tradenames EUDRAGIT® L, EUDRAGIT® S, EUDRAGIT® RL, EUDRAGIT® RS coating materials and mixtures thereof. A gastro-resistant coat can, for example, allow the pharmaceutical compositions to remain intact in the harsh low pH environment of the stomach and to dissolve when the tablet reaches the desired section of intestine.

Furthermore, one or more protective coatings consisting of e.g. HPMC or talc might be applied between tablet core and gastro-resistant coat. As used herein, “tablet core” is understood to mean a pharmaceutical composition according to the present disclosure without any external coating.

The gastro-resistant coating may be present in the pharmaceutical compositions disclosed herein in an amount ranging from about 0 mg to about 200 mg, for instance from about 1 mg to about 150 mg, for example, from about 20 mg to about 100 mg, such as from about 5 mg to about 80 mg. In certain embodiments, the gastro-resistant coating is expressed as percent by weight of the solid dosage form, for instance about 0.0% to about 10% by weight of the solid dosage form, for instance, about 0.01% to about 10% by weight of the solid dosage form, for example, about 0.03% to about 10% by weight of the solid dosage form, such as about 0.1% to about 8% by weight of the solid dosage form. In certain embodiments, the gastro-resistant coating is about 0.3% to about 0.7%, such as about 0.6%, and in some embodiments, about 0.64% by weight of the solid dosage form. In other embodiments, the gastro-resistant coating is about 5.0%, or about 6.0%, or about 7.0%, and in some embodiments, about 0.64% by weight of the solid dosage form. Pharmaceutical compositions comprising protective coatings may comprise about 0 mg to about 200 mg of a protective coating.

### Examples of Compositions

In one or more embodiment of the present disclosure, the pharmaceutical composition includes but is not limited to the examples shown in Table 2 and Table 3 below.

TABLE 2
Examples of compositions for mini-tablet, tablet or capsule dosage forms (mg)

| Examples | 1 | 2 | 3 | 4 | 5 | 6 | 7 |
|---|---|---|---|---|---|---|---|
| ASO | 5-40 | 5-40 | 5-40 | 5-40 | 5-40 | 5-40 | 0.5-5 |
| Sodium caprate | 700 | 650-700 | 650-700 | 650-700 | 650-700 | 570-700 | 18-200 |
| Mannitol | 200 | 150-200 | 150 | 150-200 | 0 | 250-300 | 0 |
| Microcrystalline | 0 | 0 | 0 | 0 | 0 | 83-102 | 0 |
| cellulose | | | | | | | |
| Crospovidone | 0 | 100-200 | 0 | 0 | 0 | 0 | 0 |
| Sodium | 0 | 0 | 100-200 | 0 | 0 | 0 | 0 |
| Croscaramellose | | | | | | | |
| Tartaric acid | 0 | 0 | 0 | 50-200 | 0 | 0 | 0 |
| Sodium bicarbonate | 0 | 0 | 0 | 50-200 | 0 | 0 | 0 |
| Dicalcium phosphate | 0 | 0 | 0 | 0 | 200 | 0 | 0 |
| PVP K30 | 0 | 0 | 0 | 0 | 34 | 0 | 0 |
| Silica colloidal | 9-10 | 9-10 | 9-10 | 9-10 | 9-10 | 9-10 | 0 |
| hydrated | | | | | | | |
| Sodium stearyl | 19-20 | 19-20 | 19-20 | 19-20 | 19-20 | 19-20 | 0 |
| fumarate | | | | | | | |

TABLE 3
Example of gastro-resistant coatings for solid
oral dosage forms (e.g., tablet or capsule)

| | % by |
|---|---|
| Example of Gastro-Resistant Coating | weight |
| Glycerol monostearate 40-55 type II | 0.3 |
| Methacrylic acid-Ethyl Acrylate Copolymer | 6.4 |
| (1:1) Dispersion 30% | |
| Polysorbate 80 | 0.03 |
| Triethyl citrate | 0.64 |

### Methods of Treatment by Orally Administering One or More ASOs

Methods of orally administering one or more ASOs are provided herein. In at least one embodiment, disclosed herein are methods of treating a subject comprising orally administering one or more ASOs or a pharmaceutically acceptable salt thereof and one or more permeation enhancers in a solid dosage form to a subject in need thereof.

In certain embodiments, methods are disclosed for reducing translation of the nucleic acid transcript to proteins involved in various disease processes in a subject, comprising orally administering to the subject a pharmaceutical composition comprising one or more ASOs, or a pharmaceutically acceptable salt thereof and one or more permeation enhancers. In at least one embodiment, the pharmaceutical composition comprises

In certain embodiments, methods are disclosed for treating, preventing, or ameliorating a disease associated with PCSK9 in a subject comprising orally administering to the subject a pharmaceutical composition comprising one or more ASOs, or a pharmaceutically acceptable salt thereof and one or more permeation enhancers. In at least one embodiment, the one or more ASO or a pharmaceutically acceptable salt thereof targets a PCSK9 nucleic acid and comprises the nucleobase sequence SEQ ID NO: 1. In another embodiment, the one or more permeation enhancer is sodium caprate. In yet another embodiment, the one or more ASO is ION-863633. In at least one embodiment, the one or more ASO is a sodium salt of ION-863633. In yet another embodiment, the one or more permeation enhancer is Form A sodium caprate.

Examples of diseases associated with PCSK9 treatable, preventable, and/or ameliorable with the methods provided herein include cardiovascular disease, dyslipidemia, mixed dyslipidemia, hypercholesterolemia, a reduction in LDL cholesterol, and reduction in atherogenic apolipoprotein (a) [Lp(a)].

In certain embodiments, methods are disclosed for reducing LDL-cholesterol levels in a subject, comprising orally administering to the subject a pharmaceutical composition comprising one or more ASOs, or a pharmaceutically acceptable salt thereof and one or more permeation enhancers. In at least one embodiment, the one or more ASO or a pharmaceutically acceptable salt thereof targets a PCSK9 nucleic acid and comprises the nucleobase sequence SEQ ID NO: 1. In another embodiment, the one or more permeation enhancer is sodium caprate. In yet another embodiment, the one or more ASO is ION-863633. In at least one embodiment, the one or more ASO is a sodium salt of ION-863633. In yet another embodiment, the one or more permeation enhancer is Form A sodium caprate.

Pharmaceutical compositions comprising one or more ASOs, or a pharmaceutically acceptable salt thereof and one or more permeation enhancers are also disclosed for use in the treatment of reducing LDL-cholesterol levels in a subject according to the present disclosure. In at least one embodiment, the one or more ASO or a pharmaceutically acceptable salt thereof targets a PCSK9 nucleic acid and comprises the nucleobase sequence SEQ ID NO: 1. In another embodiment, the one or more permeation enhancer is sodium caprate. In yet another embodiment, the one or more ASO is ION-863633. In at least one embodiment, the one or more ASO is a sodium salt of ION-863633. In yet another embodiment, the one or more permeation enhancer is Form A sodium caprate.

In certain embodiments, methods are disclosed for reducing Lp(a) levels in a subject, comprising orally administering to the subject a pharmaceutical composition comprising one or more ASOs, or a pharmaceutically acceptable salt thereof and one or more permeation enhancers. In at least one embodiment, the one or more ASO or a pharmaceutically acceptable salt thereof targets a PCSK9 nucleic acid and comprises the nucleobase sequence SEQ ID NO: 1. In another embodiment, the one or more permeation enhancer is sodium caprate. In yet another embodiment, the one or more ASO is ION-863633. In at least one embodiment, the one or more ASO is a sodium salt of ION-863633. In yet another embodiment, the one or more permeation enhancer is Form A sodium caprate.

Pharmaceutical compositions comprising one or more ASOs, or a pharmaceutically acceptable salt thereof and one or more permeation enhancers are also disclosed for use in reducing Lp(a) levels in a subject according to the present disclosure. In at least one embodiment, the one or more ASO or a pharmaceutically acceptable salt thereof targets a PCSK9 nucleic acid and comprises the nucleobase sequence SEQ ID NO: 1. In another embodiment, the one or more permeation enhancer is sodium caprate. In yet another embodiment, the one or more ASO is ION-863633. In at least one embodiment, the one or more ASO is a sodium salt of ION-863633. In yet another embodiment, the one or more permeation enhancer is Form A sodium caprate.

In certain embodiments, methods are disclosed for inducing LDL receptor (LDL-R) activity in a subject, comprising orally administering to the subject a pharmaceutical composition comprising one or more ASOs, or a pharmaceutically acceptable salt thereof and one or more permeation enhancers. In at least one embodiment, the one or more ASO or a pharmaceutically acceptable salt thereof targets a PCSK9 nucleic acid and comprises the nucleobase sequence SEQ ID NO: 1. In another embodiment, the one or more permeation enhancer is sodium caprate. In yet another embodiment, the one or more ASO is ION-863633. In at least one embodiment, the one or more ASO is a sodium salt of ION-863633. In yet another embodiment, the one or more permeation enhancer is Form A sodium caprate.

Pharmaceutical compositions comprising one or more ASOs, or a pharmaceutically acceptable salt thereof and one or more permeation enhancers are also disclosed for use in inducing LDL receptor (LDL-R) activity in a subject, comprising orally administering to the subject a pharmaceutical composition according to the present disclosure. In at least one embodiment, the one or more ASO or a pharmaceutically acceptable salt thereof targets a PCSK9 nucleic acid and comprises the nucleobase sequence SEQ ID NO: 1. In another embodiment, the one or more permeation enhancer is sodium caprate. In yet another embodiment, the one or more ASO is ION-863633. In at least one embodiment, the one or more ASO is a sodium salt of ION-863633. In yet another embodiment, the one or more permeation enhancer is Form A sodium caprate.

In certain embodiments, methods are disclosed for regulating LDL receptor-LDL-cholesterol homeostasis in a subject, comprising orally administering to the subject a pharmaceutical composition comprising one or more ASOs, or a pharmaceutically acceptable salt thereof and one or more permeation enhancers. In a least one embodiment, the one or more ASO or a pharmaceutically acceptable salt thereof targets a PCSK9 nucleic acid and comprises the nucleobase sequence SEQ ID NO: 1. In another embodiment, the one or more permeation enhancer is sodium caprate. In yet another embodiment, the one or more ASO is ION-863633. In at least one embodiment, the one or more ASO is a sodium salt of ION-863633. In yet another embodiment, the one or more permeation enhancer is Form A sodium caprate.

Pharmaceutical compositions comprising one or more ASOs, or a pharmaceutically acceptable salt thereof and one or more permeation enhancers are also disclosed for use in regulating LDL receptor-LDL-cholesterol homeostasis in a subject, comprising orally administering to the subject a pharmaceutical composition according to the present disclosure. In a least one embodiment, the one or more ASO or a pharmaceutically acceptable salt thereof targets a PCSK9 nucleic acid and comprises the nucleobase sequence SEQ ID NO: 1. In another embodiment, the one or more permeation enhancer is sodium caprate. In yet another embodiment, the one or more ASO is ION-863633. In at least one embodiment, the one or more ASO is a sodium salt of ION-863633. In yet another embodiment, the one or more permeation enhancer is Form A sodium caprate.

In certain embodiments, methods are disclosed for treating, preventing, or ameliorating a disease associated with PNPLA3 in a subject comprising orally administering to the subject a pharmaceutical composition comprising one or more ASOs, or a pharmaceutically acceptable salt thereof and one or more permeation enhancers. In at least one embodiment, the one or more ASO or a pharmaceutically acceptable salt thereof targets a PNPLA3 nucleic acid and comprises the nucleobase sequence SEQ ID NO: 2. In another embodiment, the one or more permeation enhancer is sodium caprate. In yet another embodiment, the one or more ASO is ION-975616. In at least one embodiment, the one or more ASO is a sodium salt of ION-975616. In yet another embodiment, the one or more permeation enhancer is Form A sodium caprate.

Examples of diseases associated with PNPLA3 treatable, preventable, and/or ameliorable with the methods provided herein include liver disease, non-alcoholic fatty liver disease (NAFLD), liver cirrhosis, hepatocellular carcinoma, alcoholic liver disease, alcoholic steatohepatitis (ASH), HCV hepatitis, chronic hepatitis, hereditary hemochromatosis, and/or primary sclerosing cholangitis.

In certain embodiments, the disease associated with PNPLA3 treatable, preventable and/or ameliorable with the methods provided herein include NAFLD, steatosis, NASH, and cirrhosis.

In certain embodiments, methods are disclosed for reducing and/or inhibiting liver damage, steatosis, liver fibrosis, liver inflammation, liver scarring or cirrhosis, liver failure, liver enlargement, elevated transaminases, or hepatic fat accumulation in a subject, comprising orally administering to the subject a pharmaceutical composition comprising one or more ASOs, or a pharmaceutically acceptable salt thereof and one or more permeation enhancers. In at least one embodiment, the one or more ASO or a pharmaceutically acceptable salt thereof targets a PNPLA3 nucleic acid and comprises the nucleobase sequence SEQ ID NO: 2. In another embodiment, the one or more permeation enhancer is sodium caprate. In yet another embodiment, the one or more ASO is ION-975616. In at least one embodiment, the one or more ASO is a sodium salt of ION-975616. In yet another embodiment, the one or more permeation enhancer is Form A sodium caprate.

Pharmaceutical compositions comprising one or more ASOs, or a pharmaceutically acceptable salt thereof and one or more permeation enhancers are also disclosed for use in the treatment of reducing and/or inhibiting liver damage, steatosis, liver fibrosis, liver inflammation, liver scarring or cirrhosis, liver failure, liver enlargement, elevated transaminases, or hepatic fat accumulation in a subject according to the present disclosure. In at least one embodiment, the one or more ASO or a pharmaceutically acceptable salt thereof targets a PNPLA3 nucleic acid and comprises the nucleobase sequence SEQ ID NO: 2. In another embodiment, the one or more permeation enhancer is sodium caprate. In yet another embodiment, the one or more ASO is ION-975616. In at least one embodiment, the one or more ASO is a sodium salt of ION-975616. In yet another embodiment, the one or more permeation enhancer is Form A sodium caprate.

In certain embodiments, methods are disclosed for reducing and/or inhibiting liver damage, steatosis, liver fibrosis, liver inflammation, liver scarring or cirrhosis, liver failure, liver enlargement, elevated transaminases, or hepatic fat accumulation in a subject, comprising orally administering to the subject a pharmaceutical composition comprising one or more ASOs, or a pharmaceutically acceptable salt thereof and one or more permeation enhancers. In at least one embodiment, the one or more ASO or a pharmaceutically acceptable salt thereof targets a PNPLA3 nucleic acid and comprises the nucleobase sequence SEQ ID NO: 2. In another embodiment, the one or more permeation enhancer is sodium caprate. In yet another embodiment, the one or more ASO is ION-975616. In at least one embodiment, the one or more ASO is a sodium salt of ION-975616. In yet another embodiment, the one or more permeation enhancer is Form A sodium caprate.

Pharmaceutical compositions comprising one or more ASOs, or a pharmaceutically acceptable salt thereof and one or more permeation enhancers are also disclosed for use in reducing and/or inhibiting liver damage, steatosis, liver fibrosis, liver inflammation, liver scarring or cirrhosis, liver failure, liver enlargement, elevated transaminases, or hepatic fat accumulation in a subject according to the present disclosure. In at least one embodiment, the one or more ASO or a pharmaceutically acceptable salt thereof targets a PNPLA3 nucleic acid and comprises the nucleobase sequence SEQ ID NO: 2. In another embodiment, the one or more permeation enhancer is sodium caprate. In yet another embodiment, the one or more ASO is ION-975616. In at least one embodiment, the one or more ASO is a sodium salt of ION-975616. In yet another embodiment, the one or more permeation enhancer is Form A sodium caprate.

In certain embodiments, methods are disclosed for reducing and/or inhibiting liver damage, steatosis, liver fibrosis, liver inflammation, liver scarring or cirrhosis, liver failure, liver enlargement, elevated transaminases, or hepatic fat accumulation in a subject, comprising orally administering to the subject a pharmaceutical composition comprising one or more ASOs, or a pharmaceutically acceptable salt thereof and one or more permeation enhancers. In at least one embodiment, the one or more ASO or a pharmaceutically acceptable salt thereof targets a PNPLA3 nucleic acid and comprises the nucleobase sequence SEQ ID NO: 2. In another embodiment, the one or more permeation enhancer is sodium caprate. In yet another embodiment, the one or more ASO is ION-975616. In at least one embodiment, the one or more ASO is a sodium salt of ION-975616. In yet another embodiment, the one or more permeation enhancer is Form A sodium caprate.

Pharmaceutical compositions comprising one or more ASOs, or a pharmaceutically acceptable salt thereof and one or more permeation enhancers are also disclosed for use in reducing and/or inhibiting liver damage, steatosis, liver fibrosis, liver inflammation, liver scarring or cirrhosis, liver failure, liver enlargement, elevated transaminases, or hepatic fat accumulation in a subject, comprising orally administering to the subject a pharmaceutical composition according to the present disclosure. In at least one embodiment, the one or more ASO or a pharmaceutically acceptable salt thereof targets a PNPLA3 nucleic acid and comprises the nucleobase sequence SEQ ID NO: 2. In another embodiment, the one or more permeation enhancer is sodium caprate. In yet another embodiment, the one or more ASO is ION-975616. In at least one embodiment, the one or more ASO is a sodium salt of ION-975616. In yet another embodiment, the one or more permeation enhancer is Form A sodium caprate.

In certain embodiments, methods are disclosed for regulating liver damage, steatosis, liver fibrosis, liver inflammation, liver scarring or cirrhosis, liver failure, liver enlargement, elevated transaminases, or hepatic fat accumulation in a subject, comprising orally administering to the subject a pharmaceutical composition comprising one or more ASOs, or a pharmaceutically acceptable salt thereof and one or more permeation enhancers. In a least one embodiment, the one or more ASO or a pharmaceutically acceptable salt thereof targets a PNPLA3 nucleic acid and comprises the nucleobase sequence SEQ ID NO: 2. In another embodiment, the one or more permeation enhancer is sodium caprate. In yet another embodiment, the one or more ASO is ION-975616. In at least one embodiment, the one or more

ASO is a sodium salt of ION-975616. In yet another embodiment, the one or more permeation enhancer is Form A sodium caprate.

Pharmaceutical compositions comprising one or more ASOs, or a pharmaceutically acceptable salt thereof and one or more permeation enhancers are also disclosed for use in regulating liver damage, steatosis, liver fibrosis, liver inflammation, liver scarring or cirrhosis, liver failure, liver enlargement, elevated transaminases, or hepatic fat accumulation in a subject, comprising orally administering to the subject a pharmaceutical composition according to the present disclosure. In a least one embodiment, the one or more ASO or a pharmaceutically acceptable salt thereof targets a PNPLA3 nucleic acid and comprises the nucleobase sequence SEQ ID NO: 2. In another embodiment, the one or more permeation enhancer is sodium caprate. In yet another embodiment, the one or more ASO is ION-975616. In at least one embodiment, the one or more ASO is a sodium salt of ION-975616. In yet another embodiment, the one or more permeation enhancer is Form A sodium caprate.

Subjects that can be orally administered the one or more ASO or a pharmaceutically acceptable salt thereof and one or more permeation enhancer according to the various methods described herein include mammals, for example, humans, dogs, cats, primates, etc. In at least one embodiment, the subject is a human.

In certain embodiments, the pharmaceutical compositions disclosed herein can be administered orally once per day. In certain embodiments, the pharmaceutical formulation is administered orally twice per day.

### EXAMPLES

While certain pharmaceutical compositions and methods described herein have been described with specificity in accordance with certain embodiments, the following examples serve only to illustrate the pharmaceutical compositions described herein and are not intended to limit the same. Each of the references recited in the present application is incorporated herein by reference in its entirety. It will be appreciated that where typical or exemplified process conditions (i.e., reaction temperatures, times, solvents, pressures, etc.) are given, other process conditions can also be used unless otherwise stated. Optimum reaction conditions may vary with the particular reactants or solvents used, but such conditions may be determined by one skilled in the art.

### Example 1: Preparation of Tablets Comprising Form a Sodium Caprate and PCSK9 ASO (ION 863633)

Preparation of Form A Sodium Caprate

Decanoic acid was dissolved in methanol followed by the addition of solid sodium bicarbonate (IPC pH control). The suspension was heated to reflux. The resulting solution was then cooled down to ambient temperature to crystallize. The solids were collected by centrifugation, dried and finally milled.

Preparation of Tablet Comprising Form A Sodium Caprate and PCSK9 ASO (ION 863633)

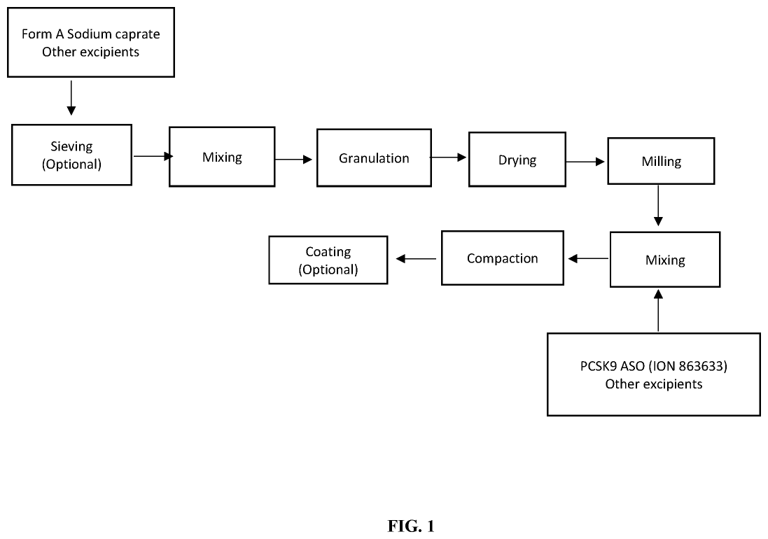

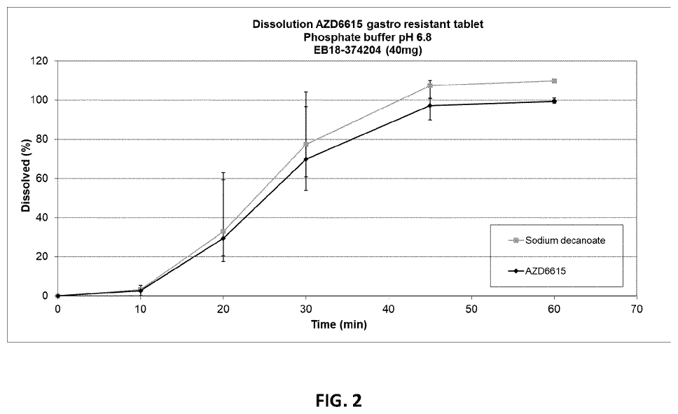

The required amount of sodium caprate Form A and mannitol was weighed into a 60L high shear mixer. The two components were dry mixed for 1 minute in the high shear mixer at an impeller speed of 160 rpm. During continuous mixing in the high shear mixer, ethanol was added until appropriate degree of granulation was reached. After granulation, the wet granules were transferred into a fluid bed dryer and dried at an inlet temperature of 70° C. until pre-defined loss on drying of the granules was reached (<1.8%). The dried granules were milled through a cone mill with a screen size of 1 mm. The milled granules were then blended in a 160 L diffusion mixer, with PCSK9 ASO (ION 863633), silica colloidal hydrated (through a screen size of 0.5 mm) and sodium stearyl fumarate (through a screen size of 0.5 mm) for 11 minutes at a rotational speed of 30 rpm. The final blended granules were compacted to uncoated core tablets using an eight-station rotary tablet press. The uncoated tablets were then coated with a gastric-resistant coating (sieved through a screen size 0.25 mm) in a pan coater (drum size 5 kg) at an inlet temperature of 50° C. to 65° C. The process flowchart is schematically illustrated in FIG. 1. The dissolution profile of tablets comprising Form A sodium caprate and PCSK9 ASO (ION 863633) is illustrated in FIG. 2. The graph shows that there is a concomitant release (dissolution) of the ASO (AZD6615 is PCSK9 ASO (ION 863633) in this example) and Form A sodium caprate from tablets at the same time at a pH 6.8 (mimicking the small intestinal condition) which allows for maximum absorption enhancement.

### Example 2: Plasma vs Liver Exposure of GalNac ASO in Oral Dosing in the Dog

Plasma and liver exposure were measured upon daily oral dosing of a tablet containing 700 mg sodium caprate formulated with either 3 or 20 mg PCSK9 ASO (ION 863633) for 7 or 28 days in beagle dogs. Once daily SC administration of 1 mg/kg was used as control. The plasma bioavailability is based on the area under the plasma concentration-time curve (AUC) over 24 h, while the tissue bioavailability is based on samples taken 24 h after last dose. See Table 4 below for plasma vs liver exposure of

TABLE 4
Plasma vs. liver exposure of PCSK9 ASO (ION 863633) in oral dosing in dogs. The plasma bioavailability is
based on AUC over 24 h, while the tissue bioavailability is based on samples taken 24 h after last dose.

| | | | Plasma | Liver | | | |
|---|---|---|---|---|---|---|---|
| | Daily | | AUC0-t Mean | exposure 24 h | | Plasma | Liver |
| Route | dose | Time | (ng/mL × | after last dose | Ratio | bioavailability* | bioavailability* |
| Formulation | (mg) | (day) | h; ±SEM) | (μg/g; ±SEM) | (plasma/liver) | (%; ±SEM) | (%; ±SEM) |
| SC (n = 2) | 1 | 7 | 60.2 ± N/A | 10.8 ± N/A | 5.6 | N/A | N/A |
| Solution | | | | | | | |
| oral (n = 5) | 20 | 7 | 18.3 ± 5.6 | 15.0 ± 3.6 | 1.2 | 1.5 ± 0.46 | 7.0 ± 0.74 |
| tablet | | | | | | | |
| SC (n = 2) | 1 | 28 | 68.0 ± N/A | 24.4 ± N/A | 2.8 | N/A | N/A |
| Solution | | | | | | | |
| oral (n = 5) | 3 | 28 | 4.44 ± 0.62 | 5.14 ± 1.6 | 0.86 | 2.2 ± 0.30 | 7.0 ± 1.0 |
| tablet | | | | | | | |
| oral (n = 5) | 20 | 28 | 23.5 ± 2.2 | 36.0 ± 14 | 0.65 | 1.7 ± 0.16 | 7.4 ± 1.2 |
| tablet | | | | | | | |

*versus daily SC administration of 1 mg for the same duration

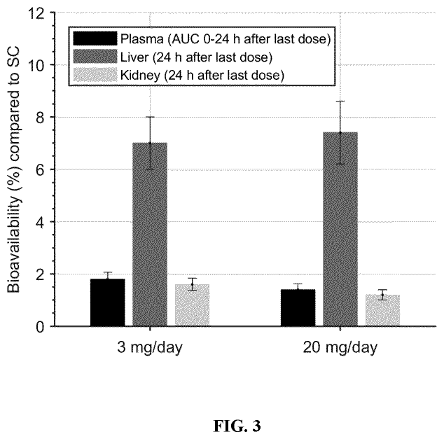

Results: The plasma/liver ratio is shifted between subcutaneous and oral dosing towards a higher relative liver exposure after oral dosing. Data support that direct delivery of an ASO to the portal vein via oral delivery gains additional benefits from first pass extraction, i.e., the compound is distributed more efficiently to the target tissue/liver versus plasma (as seen in the Table 4 above). Selective uptake by the liver is further supported by a limited kidney bioavailability of 1-2%, in the same range as plasma bioavailability as shown in FIG. 3.

Experimental: PCSK9 ASO (ION- 863633) was dosed daily to 19 male Beagle dogs as an oral tablet or as a SC solution, for 7 or 28 days. The oral doses were 3 (n=5) or 20 (n=10) mg/day where half of the high dose animals were terminated after 7 days of dosing. The SC dose was 1 mg/day (n=4), and half of these animals were also terminated after 7 days of dosing. Thirty minutes or less prior to tablets administration a solution of 0.1 M HCl/KCl was administered by oral gavage using a disposable catheter attached to a plastic syringe at a volume of 30 mL. At administration, the tablet was placed as far back into the throat as possible (using fingers) followed by a tap water flush (10 mL) administered into the mouth using a syringe to encourage swallowing of the tablet. The SC dose was injected into the scapular and mid-dorsal areas. The plasma exposure was evaluated on day 1, 7 and 28 by taking blood samples from the jugular vein up to 24 h post dose. The solution for SC administration contained PCSK9 ASO (ION 863633) 1 mg/mL in PBS pH 7.4 (10 mM phosphate+150 mM NaCl).

### Example 3: Rat Data Supporting Beneficial Effect From First Pass Extraction

Since PCSK9 ASO (ION 863633) is not active in rodents we also used a rat-specific GalNAc-conjugated Malat-1 ASO (16-mer cEt GalNAc3-conjugate targeting Malat-1; ION-704361) which is of the same chemistry as PCSK9 ASO (ION 863633) to confirm target engagement in form of mRNA knockdown after intrajejunal (IJ) administration of solutions containing ASO and permeation enhancer. See Table 5 below.

TABLE 5
Liver bioavailability and productive liver uptake measured by
mRNA knockdown for single dose PCSK9 ASO (ION 863633)
or ION-704361 administered to rats either as SC or IJ administration.
The tissue bioavailability is based on samples taken 48 h after last dose.

| | Liver bioavailability* | Productive bioavailability** |
|---|---|---|
| Compound | (%; 5th and 95th percentiles) | (%; 5th and 95th percentiles) |
| PCSK9 ASO | 5.3 (4.2-6.2) | |
| (n = 16) | | |
| ION-704361 | 5.0 (4.3-5.9) | 29 (10-100) |
| (n = 16) | | |

*based on liver tissue exposures for IJ versus SC administration
**target engagement (mRNA knock down) for IJ versus SC administration

Results: Data indicates that target tissue cell productive uptake is even more pronounced after IJ administration of solutions compared to looking at liver bioavailability based on total liver tissue exposures. This supports the hypothesis that oral dosing and first pass extraction is beneficial for the uptake to the liver for GalNAc ASOs.

Experimental: We used ASOs with similar chemistries (16-mer cEt GalNAc3-conjugated) targeting rat Malat-1 (ION-704361) or human PCSK9. The rat-specific ASO allowed for both liver exposure and target-engagement measurement, while only liver exposure was observed for the human-specific PCSK9 ASO (ION 863633). Male Sprague Dawley rats, 6 to 8 weeks and weighing 200-250 g, with surgically implanted IJ catheter were exposed to ION-704361 or PCSK9 ASO (ION 863633), using a single IJ or SC dose (n=4 per group). All animals were sacrificed at 48 h. Sodium caprate at a dose of 300 mg/kg was used as permeability enhancer for IJ administration. The ASOs and sodium caprate was administered as solutions. Plasma and liver tissue samples were collected at 48 h. Hybridization ELISA was performed for plasma, and LC-MS/MS analysis for tissue. ION-863633 and ION-704361 plasma LLOQ: 0.15 nM. Unconjugated PCSK9 ASO (ION 848833) and ION-704361 tissue LLOQ: 0.054 and 0.0269 μg/g respectively. Immunohistochemistry, Hematoxylin and eosin staining, and in situ hybridization was conducted for all tissues. Malat-1 mRNA expression and knockdown in liver was assessed by real-time PCR, and relative levels with respect to the SC control group were reported.

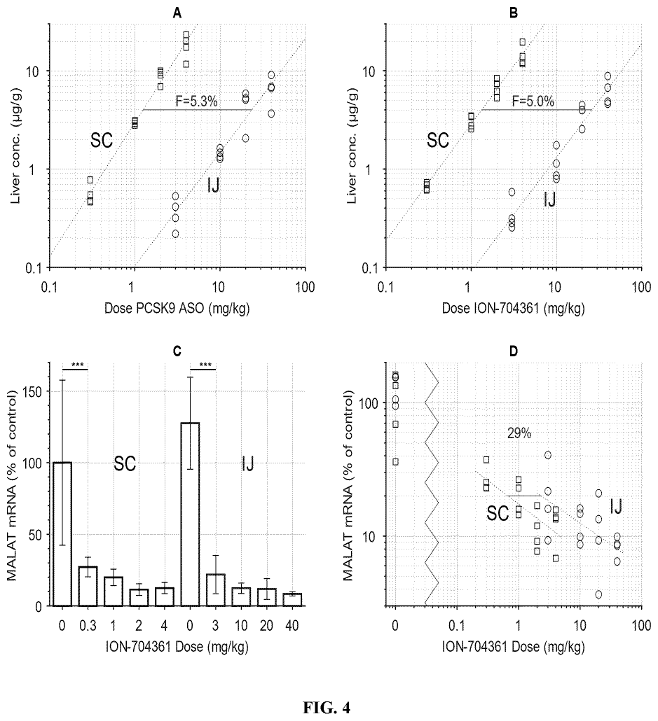

The generated data were analysed in the following ways: Bioavailability of IJ versus SC was based on linear regression of data for each administration route separately and calculated at a therapeutically relevant level of liver exposure or target engagement. The linear regression is indicated by the dotted lines and the level for the bioavailability calculation by the solid lines in FIG. 4A, B and D. In FIG. 4C, error bars denote standard deviation, and the horizontal bars indicate the significant differences between treatments (***p≤0.005) for Tukey's honestly significant difference test. The productive bioavailability of IJ compared to SC was 29% (10%, 100%; 5th and 95th percentiles). In FIG. 4C, the x-axis is broken to allow the control groups at dose level 0 to be included. FIG. 4D depicts individual data from FIG. 4C plotted versus dose. Uncertainty of parameter estimates was determined by bootstrapping, sampling single measurements randomly with replacement within each experiment.

### Example 4: Prospective Human Study

Clinical trials of the PCSK9 ASO (ION 863633) as a solid dosage form are planned in patients to establish safe and tolerable doses for the solid dosage form. Plasma exposure and PCSK9 levels (correlates to liver exposure) will be measured upon daily oral dosing of 1-3 tablets each containing 700 mg sodium caprate formulated with either 5, 10, 20 or 40 mg PCSK9 ASO (ION-863633) after single dose as well as after repeated once daily dosing for 28 days in human subjects. The tissue half-life of the PCSK9 ASO is in the order of two weeks. Therefore, a single dose is not expected to decrease PCSK9 plasma levels significantly, while 28 days of dosing will lead to a build-up of drug in the liver and a pronounced PCKS9 and LDL reduction.

### Example 5: Preparation of Tablets Comprising Form a Sodium Caprate And PNPLA3 ASO (ION 975616)

Preparation of Form A Sodium Caprate

Methanol (234 L) and water (12.3 L) was added to pre-melted decanoic acid (24.6 kg, 143 mols) and sodium bicarbonate (11.0 kg, 131 mols). The reaction mixture was heated at 65±5° C. and stirred for at least 16 hours. When the reaction was finished (decanoic acid ≤3 mg/mL) the reaction solution was filtered through a polish filter. The temperature was adjusted 50±5° C., the mixture was cooled (5° C./h) to 27±3° C. and tent-butyl methyl ether (TBME, 492 liters) was added. The temperature was adjusted to 25±5° C. and stirred for at least 12 hours. The solids were collected by centrifugation and washed with TMBE (123 liters). The solids were dried under reduced pressure at 50±5° C. for at least 12 hours (Loss on Drying, LoD≤2.0%) to afford 20.99 kg of the target product, yield 81.3%, LoD 0.6%.

### Example 5A (formulation 1): Tablets Comprising Form A Sodium Caprate, Microcrystalline Cellulose, and PNPLA3 ASO (ION 975616)

1200 g of sodium caprate Form A was added to an intensive blending equipment and dry mixed, impeller speed 250 rpm and chopper speed 1500 rpm, for 1 min before addition of 600 g of absolute ethanol (30 mL/min). After complete addition the resulting material was mixed for an additional 1 min. The material was dried overnight in a fume cupboard at ambient temperature until <LoD 1.5%. The dried material was milled using a Quadro comil equipped with a 0.061″ grater screed and an angular impeller at 2000 rpm

Sodium caprate Form A (45.7 g), micro crystalline cellulose (Avicel PH-102, 2.5 g) and PNPLA3 ASO (ION 975616) (1.8 g @ 77.9%) (passed through a 250 um sieve) were pre-blended, by hand with a spoon, in a 1 L metal vessel for 1 min. Additional blending was done in a turbula mixer for 10 min. The material was compacted “as is” in a tablet press with a tensile strength of 1.4 MPa (766 mg, 8.5 mm×17 mm).

### Example 5B (formulation 2): Tablets Comprising Form A Sodium Caprate, Mannitol, Microcrystalline Cellulose, Sodium Stearyl Fumarate and PNPLA3 ASO (ION 975616)

Sodium caprate Form A (7700 g) and mannitol ((Pearlitol 100SD, 3300 g) were weighed into a 60 L high shear mixer. The two components were dry mixed for 3 minutes with an impeller speed of 140 rpm. During continuous mixing ethanol (4050 g) was added until appropriate degree of granulation was reached. After granulation, the wet granules were transferred into a fluid bed dryer and dried at an inlet temperature of 70° C. until pre-defined loss on drying of the granules was reached (<1.8%). The dried granules were milled through a cone mill with a screen size of 1.27 mm. LoD after milling was 0.5%-w/w.

The dried granules from above (47.8 g), micro crystalline cellulose (Avicel PH-102, 2.5 g), silicon dioxide (Syloid 244FP, 0.5 g) and PNPLA3 ASO (ION 975616) (1.25 g @ 77.9%), passed through a 250 um sieve, were pre-blended in a 1 L metal vessel for 1 min by hand with a spoon, additional blending was done in a turbula mixer for 10 min. Sodium stearyl fumarate (1.0 g) was added to about 10 g the resulting mixture above and pre-blended for 1 min by hand with a spoon, the remaining amount of the mixture above was added and additional blending was done in a by hand with a spoon for 2 min. The material was compacted “as is” in a tablet press with a tensile strength of 2.0 MPa (1117 mg, 9.5 mm×20 mm).

### Example 6: In Vivo Monkey Data After Repeated Oral Administration of PCSK9 ASO (ION 863633)

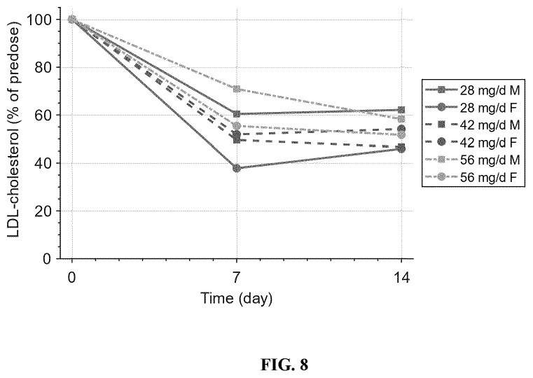

The tolerability of high doses of PCSK9 ASO (ION 863633) tablets in healthy cynomolgus monkey following daily oral administration was investigated in a 14-day study. PCSK9 ASO (ION 863633) formulated in an enteric-coated tablet with sodium caprate (14 mg PCSK9 ASO (ION 863633)+500 mg sodium caprate) at dose levels of 2, 3 or 4 tablets per day. FIG. 8 illustrates that plasma LDL-cholesterol was reduced by 45-50% at day 14 (average predose level was 55±2.7 mg/dL (mean±SEM)). The reduction was independent of dose level, reflecting the high doses used in the study. No adverse effects on clinical observations, body weights, food consumption, haematology, coagulation, clinical chemistry, organ weights and gross and microscopic pathology. Isolated instances of vomit were observed immediately after dosing in all animals dosed at ≥42 mg/day. The cause of the emesis was considered to be procedural in origin, since there was no evidence of the tablets being present in the vomit.

1. A pharmaceutical composition comprising
A) one or more ASO or a pharmaceutically acceptable salt thereof;
B) one or more permeation enhancer;
C) one or more optional pharmaceutically acceptable excipient; and
D) one or more optional coating.

2. The pharmaceutical composition of claim 1, wherein the one or more ASO or a pharmaceutically acceptable salt thereof comprises at least one GalNAc conjugate.

3. The pharmaceutical composition of claim 1, wherein the one or more ASO or a pharmaceutically acceptable salt thereof targets a PCSK9 nucleic acid and has a nucleobase sequence comprising SEQ ID NO: 1.

4. The pharmaceutical composition of claim 3, wherein the ASO is a compound chosen from ION 863633 and ION 848833 or a pharmaceutically acceptable salt thereof.

5. The pharmaceutical composition of claim 1, wherein the one or more permeation enhancer is chosen from medium chain fatty acids and their salts.

6. The pharmaceutical composition of claim 5, wherein the one or more permeation enhancer is chosen from sodium caprylate, sodium caprate, sodium laurate, sodium myristate, sodium palmitate and sodium stearate.

7. The pharmaceutical composition of claim 6, wherein the one or more permeation enhancer is sodium caprate.

8. The pharmaceutical composition of claim 7, wherein the one or more permeation enhancer is Form A sodium caprate.

9. The pharmaceutical composition of claim 1, wherein the composition comprises
A) an ASO chosen from ION 863633 and ION 848833 or a pharmaceutically acceptable salt thereof present in an amount within the range from about 1 to about 100 mg;
B) Form A sodium caprate present in an amount within the range from about 10 to about 1000 mg;
C) one or more pharmaceutically acceptable excipient present in an amount ranging from about 0 to about 600 mg; and
D) one or more optional coating present in an amount within the range from about 0 mg to about 100 mg.

10. A method of treating, preventing, or ameliorating a disease associated with PCSK9 in a subject comprising administering to the subject a pharmaceutical composition according to claim 1.

11. The method of claim 10, wherein the disease is a cardiovascular disease chosen from dyslipidemia, mixed dyslipidemia, and hypercholesterolemia.

12. Use of a pharmaceutical composition according to claim 1, for treating, preventing, or ameliorating a disease associated with PCSK9.

13. The use of claim 12, wherein the disease is a cardiovascular disease chosen from dyslipidemia, mixed dyslipidemia, and hypercholesterolemia.

14. The pharmaceutical composition of claim 1, wherein the one or more ASO or a pharmaceutically acceptable salt thereof targets a PNPLA3 nucleic acid and has a nucleobase sequence comprising SEQ ID NO: 2.

15. The pharmaceutical composition of claim 14, wherein the ASO is a compound chosen from ION 975616 and ION 916333 or a pharmaceutically acceptable salt thereof.

16. The pharmaceutical composition of claim 1, wherein the composition comprises
A) an ASO chosen from ION 975616 and ION 916333 or a pharmaceutically acceptable salt thereof present in an amount within the range from about 1 to about 100 mg;
B) Form A sodium caprate present in an amount within the range from about 10 to about 1000 mg;
C) one or more pharmaceutically acceptable excipient present in an amount ranging from about 0 to about 600 mg; and
D) one or more optional coating present in an amount within the range from about 0 mg to about 100 mg.

17. A method of treating, preventing, or ameliorating a disease associated with PNPLA3 in a subject comprising administering to the subject a pharmaceutical composition according to claim 1.

18. The method of claim 17, wherein the disease is chosen from liver disease, non-alcoholic fatty liver disease (NAFLD), liver cirrhosis, hepatocellular carcinoma, alcoholic liver disease, alcoholic steatohepatitis (ASH), HCV hepatitis, chronic hepatitis, hereditary hemochromatosis, and/or primary sclerosing cholangitis.

19. Use of a pharmaceutical composition according to claim 1, for treating, preventing, or ameliorating a disease associated with PNPLA3.

20. The use of claim 19, wherein the disease is chosen from liver disease, non-alcoholic fatty liver disease (NAFLD), liver cirrhosis, hepatocellular carcinoma, alcoholic liver disease, alcoholic steatohepatitis (ASH), HCV hepatitis, chronic hepatitis, hereditary hemochromatosis, and/or primary sclerosing cholangitis.
